(19) United States
(12) Patent Application Publication
(10) Pub. No.: US 2026/0167967 A1
(43) Pub. Date: Jun. 18, 2026

(54) COMPOSITIONS, SYSTEMS, AND METHODS TARGETING GASDERMIN E

(71) Applicant: University of Virginia Patent Foundation, Charlottesville, VA (US)

(72) Inventors: Jayakrishna Ambati, Charlottesville, VA (US); Shao-bin Wang, Charlottesville, VA (US)

(21) Appl. No.: 19/417,660

(22) Filed: Dec. 12, 2025

Related U.S. Application Data
(60) Provisional application No. 63/733,165, filed on Dec. 12, 2024.

(51) Int. Cl. C12N 15/113; A61K 31/20; A61K 31/22; A61K 31/30; A61K 31/575; A61K 38/08; A61P 27/02; C12N 15/11

(52) U.S. Cl. CPC C12N 15/1135; A61K 31/20; A61K 31/22; A61K 31/30; A61K 31/575; A61K 38/08; A61P 27/02; C12N 15/111; C12N 2310/14; C12N 2310/20; C12N 2310/531

(57) ABSTRACT

Described herein are compositions, pharmaceutical compositions, methods, and kits relating to knocking down, inhibiting, or otherwise reducing the activity and/or expression of Gasdermin E. Methods as described herein can be indicated, for example, in subjects having or suspected of having, for example, Traumatic Optic Neuropathy (TON), Glaucoma, juvenile glaucoma, Leber's Hereditary Optic Neuropathy, Optic Neuritis, Ischemic Optic Neuropathy, Diabetic Retinopathy, Retinal Occlusion, Anterior Ischemic Optic Neuropathy (AION), Infectious Neuropathies, Neuromyelitis optica (NMO), Behçet's disease, optic nerve hypoplasia, Central or branch retinal artery occlusion (CRAO, BRAO), Radiation Optic Neuropathy, Optic nerve glioma, Age-related macular degeneration (AMD).

### CROSS-REFERENCE TO RELATED APPLICATION[S]

This application claims priority to, and the benefit of, U.S. Provisional Patent Application Ser. No. 63/733,165, entitled “COMPOSITIONS, SYSTEMS, AND METHODS TARGETING GASDERMIN E” and filed on Dec. 12, 2024, the entire contents of which are incorporated herein by reference as if set forth in its entirety.

### SEQUENCE LISTING

The instant application contains a Sequence Listing which has been submitted electronically in .XML format and is hereby incorporated by reference in its entirety. Said .XML copy, created on Dec. 11, 2025, is named “222117_1730_Sequence_Listing.xml” and is 42,900 bytes in size.

### BACKGROUND

Traumatic optic neuropathy (TON) is the leading neuro-ophthalmic injury in U.S. soldiers, often causing vision loss or permanent blindness, significantly impacting veterans' quality of life. Current treatments, including high-dose corticosteroids and surgical decompression are largely ineffective due to limited understanding of the fundamental mechanism underlying retinal ganglion cell (RGC) death. Accordingly, there is a need to address the aforementioned deficiencies and inadequacies.

### SUMMARY

Described herein are compositions, pharmaceutical compositions, methods, methods of treatment, and kits relating to knocking down or otherwise reducing Gasdermin E activity.

Described herein are compositions for knocking down or otherwise reducing Gasdermin E activity. In an embodiment, a composition for knocking down or otherwise reducing Gasdermin E activity can comprise one or more nucleotides targeting Gasdermin E, one or more small molecules targeting or otherwise selective for Gasdermin E, or any combination of any thereof. In embodiments, the one or more nucleotides targeting Gasdermin E are one or more anti-Gasdermin E shRNAs, siRNAs, or CRISPRi sgRNAs.

In embodiments, the one or more anti-Gasdermin E shRNAs can comprise, consist essentially of, or consist of any one or more of SEQ ID NOs: 1-19, or one or more nucleotides with at least 75%, at least 80%, at least 85%, at least 90%, at least 95%, at least 96%, at least 97%, at least 98%, or at least 99% sequence identity with any one or more of SEQ ID NOs:1-19, individually or in any combination of any thereof.

In embodiments, the one or more anti-Gasdermin E siRNAs can comprise, consist essentially of, or consist of any one or more of SEQ ID NOs: 20-29, or one or more nucleotides with at least 75%, at least 80%, at least 85%, at least 90%, at least 95%, at least 96%, at least 97%, at least 98%, or at least 99% sequence identity with any one or more of SEQ ID NOs:20-29, individually or in any combination of any thereof.

In embodiments, the one or more anti-Gasdermin E CRISPRi sgRNAs can comprise, consist essentially of, or consist of any one or more of SEQ ID NOs: 31-40, or one or more nucleotides with at least 75%, at least 80%, at least 85%, at least 90%, at least 95%, at least 96%, at least 97%, at least 98%, or at least 99% sequence identity with any one or more of SEQ ID NOs:31-40, individually or in any combination of any thereof.

In embodiments, the one or more small molecules targeting or otherwise selective for Gasdermin E can comprise any one or more of CuET (copper diethyldithiocarbamate), Disulfiram 20-hydroxyecdysone, Ac-DMPD-CMK, Ac-DMLD-CMK, Dimethyl Fumarate (DMF), or 2-bromopalmitate (2-BP), individually or in any combination of any thereof.

Described herein are pharmaceutical compositions comprising any composition comprising any one or more shRNAs, siRNAs, sgRNAs, or small molecules, individually or in any combination of any thereof, and a pharmaceutically-acceptable carrier.

Described herein are methods of reducing Gasdermin E activity. In embodiments, methods of reducing Gasdermin E activity can comprise administering any composition described herein to a subject in need thereof. In embodiments, subject in need thereof is a subject having or suspected of having one or more of Traumatic Optic Neuropathy (TON), Glaucoma, juvenile glaucoma, Leber's Hereditary Optic Neuropathy, Optic Neuritis, Ischemic Optic Neuropathy, Diabetic Retinopathy, Retinal Occlusion, Anterior Ischemic Optic Neuropathy (AION), Infectious Neuropathies, Neuromyelitis optica (NMO), Behçet's disease, optic nerve hypoplasia, Central or branch retinal artery occlusion (CRAO, BRAO), Radiation Optic Neuropathy, Optic nerve glioma, or Age-related macular degeneration (AMD), individually or in any combination of any thereof. In an embodiment, the subject in need thereof is a subject having or suspected of having Traumatic Optic Neuropathy (TON).

In embodiments, a composition can be administered in an effective amount (i.e., a therapeutically effective amount) to reduce Gasdermin E activity in the subject from a first level to a second level, wherein the first level is a higher activity than the second level.

Described herein are methods of reducing neuronal cell death. In an embodiment, a method of reducing neuronal cell death can comprise administering a composition described herein to a subject in need thereof. In embodiments, the subject in need thereof is a subject having or suspected of having one or more of Traumatic Optic Neuropathy (TON), Glaucoma, juvenile glaucoma, Leber's Hereditary Optic Neuropathy, Optic Neuritis, Ischemic Optic Neuropathy, Diabetic Retinopathy, Retinal Occlusion, Anterior Ischemic Optic Neuropathy (AION), Infectious Neuropathies, Neuromyelitis optica (NMO), Behçet's disease, optic nerve hypoplasia, Central or branch retinal artery occlusion (CRAO, BRAO), Radiation Optic Neuropathy, Optic nerve glioma, or Age-related macular degeneration (AMD), individually or in any combination of any thereof. In an embodiment, the subject in need thereof is a subject having or suspected of having Traumatic Optic Neuropathy (TON).

In an embodiment, the composition is administered in an amount effective to reduce neuronal cell death in the subject from a first level to a second level, wherein the second level is a lower amount of neuronal cell death than the first level. In an embodiment, the neuronal cell death comprises retinal ganglion cell death in the subject.

Described herein are methods of reducing axonal degeneration. In an embodiment, a method of reducing axonal degeneration can comprise administering a composition described herein to a subject in need thereof. In embodiments, the subject in need thereof is a subject having or suspected of having one or more of Traumatic Optic Neuropathy (TON), Glaucoma, juvenile glaucoma, Leber's Hereditary Optic Neuropathy, Optic Neuritis, Ischemic Optic Neuropathy, Diabetic Retinopathy, Retinal Occlusion, Anterior Ischemic Optic Neuropathy (AION), Infectious Neuropathies, Neuromyelitis optica (NMO), Behçet's disease, optic nerve hypoplasia, Central or branch retinal artery occlusion (CRAO, BRAO), Radiation Optic Neuropathy, Optic nerve glioma, or Age-related macular degeneration (AMD), individually or in any combination of any thereof. In an embodiment, the subject in need thereof is a subject having or suspected of having Traumatic Optic Neuropathy (TON). In an embodiment, the axonal degeneration comprises retinal ganglion cell axonal degeneration.

In embodiments, the composition can be administered in an effective amount to reduce axonal degeneration in the subject from a first level to a second level, wherein the second level is a lower amount of axonal degeneration than the first level.

Described herein are methods of treating a disorder with retinal ganglion cell death and/or axonal degeneration. In embodiments, a method treating a disorder with retinal ganglion cell death and/or axonal degeneration can comprise administering a composition described herein to a subject in need thereof. In embodiments, the subject in need thereof is a subject having or suspected of having one or more of Traumatic Optic Neuropathy (TON), Glaucoma, juvenile glaucoma, Leber's Hereditary Optic Neuropathy, Optic Neuritis, Ischemic Optic Neuropathy, Diabetic Retinopathy, Retinal Occlusion, Anterior Ischemic Optic Neuropathy (AION), Infectious Neuropathies, Neuromyelitis optica (NMO), Behçet's disease, optic nerve hypoplasia, Central or branch retinal artery occlusion (CRAO, BRAO), Radiation Optic Neuropathy, Optic nerve glioma, or Age-related macular degeneration (AMD), individually or in any combination of any thereof. In an embodiment, the subject in need thereof is a subject having or suspected of having Traumatic Optic Neuropathy (TON). In an embodiment, the axonal degeneration comprises retinal ganglion cell axonal degeneration.

In an embodiment, the treating comprises reducing the level or severity of one or more symptoms of the disorder from a first level to a second level, wherein the second level is less symptomatic than the first level.

Described herein are kits. In embodiments, a kit can comprise a composition as described herein and instructions for use. In an embodiment, the composition is provided in a dose unit form.

### BRIEF DESCRIPTION OF THE DRAWINGS

Many aspects of the disclosed devices and methods can be better understood with reference to the following drawings. The components in the drawings are not necessarily to scale, emphasis instead being placed upon clearly illustrating the relevant principles.

Moreover, in the drawings, like reference numerals designate corresponding parts throughout the several views.

FIGS. 1A-1C: Gasdermin E expression correlates with RGC vulnerability. (FIG. 1A) Classification of three survival groups (Resilient, Intermediate and Susceptible) of mouse RGC based on the pattern of cell loss across time after optic nerve crush. (FIG. 1B) Dot plot of the single cell expression levels of murine gasdermin genes (a, c, d, e) in three survival groups of RGC. (FIG. 1C) Single cell expression levels of RGC signature genes (Rbpms, Pou4f1) and Gsdme in RGCs across time after ONC. Two-way ANOVA with tukey's multiple comparisons test. *P<0.05. **P<0.01.

FIG. 2: Mouse model of traumatic optic neuropathy (TON). The RGC specific marker, RBPMS, was used to label RGCs in flat-mounted retinas at different time points after optic nerve crush. Scale bar, 10 μm.

FIGS. 3A-3D: Neuronal gasdermin E activation in RGC degeneration. (FIG. 3A) IF staining of RGC specific marker, TUJ1, cleaved gasdermin E and Rabbit IgG isotype control with retinal whole-mounts from wild-type mice at 3 days post-ONC. (FIG. 3B) IF staining of RGC specific marker, TUJ1, cleaved gasdermin E with cryosections of wild-type mice at 3 days post-ONC. (FIG. 3C) Confocal images of gasdermin E activation and pore formation in TUJ1+ RGCs at 3 days post-ONC. (FIG. 3D) Immunoblotting and densitometry analysis of nGSDME, fGSDME and GAPDH with retina lysates collected at 3 days post-ONC.

FIGS. 4A-4C: Gasdermin E activation occurs at the early stage of RGC degeneration. (FIG. 4A) Immunofluorescence staining of TuJ1 (class III beta-tubulin) and cleaved gasdermin E (nGSDME) with retina whole-mounts from wild-type mice at various time points post-ONC. (FIG. 4B) Illustration of the methods employed using Image J to quantify the nGSDME positive RGC with retinal whole-mounts. (FIG. 4C) Quantification of nGSDME+ RGC with retina collected at various time points post-ONC. N=3, One-way ANOVA with tukey's multiple comparisons test. ****P<0.0001. Scale bar, 10 μm.

FIG. 5: Inflammasome pathway is dispensable for neuronal gasdermin E activation in RGC. Immunofluorescence staining and quantification of TuJ1 (class III beta-tubulin) and cleaved gasdermin E (nGSDME) with retinal whole-mounts from NIrp3^{-/-}, NIrc4^{-/-} and Casp1^{-/-}Casp4^{-/-} mice after optic nerve crush. N=3, One-way ANOVA with tukey's multiple comparisons test. Scale bar, 10 μm.

FIG. 6: Genetic ablation of GSDME prolongs RGC survival after optic nerve injury. Immunolabeling and quantification of RGC with retinal whole-mounts at 14 days post-ONC. N=3, Unpaired t test. *P<0.05. Scale bar, 10 μm.

FIGS. 7A-7D: AAV targeting gasdermin E prolongs RGC survival and halts axon degeneration after optic nerve injury. (FIG. 7A) Experimental design to assess in vivo therapeutic efficacy of AAV-mediated Gasdermin E knockdown in treating TON. (FIG. 7B) IF staining of RGC marker Brn3a, and (FIG. 7C) quantification of RGCs in retinas treated with AAV-GPP-shCtrl or AAV-GPP-shGsdme 14 days post-ONC (N=3; unpaired t test, P<0.05). Scale bar, 100 μm. (FIG. 7D) Confocal images of anterograde GFP tracing of RGC axons in the optic nerve 14 days post-ONC. Scale bar, 100 μm.

FIG. 8 shows an embodiment of a plasmid vector that can be utilized according to aspects of the present disclosure that can be utilized to deliver a Gasdermin E-targeting nucleotide to a cell, tissue, or subject.

FIGS. 9A-9C. Knockdown of Gsdme preserves visual function. Representative electroretinogram (ERG) waveforms (FIG. 9A) and scotopic threshold response (STR) recordings from control (FIG. 9B) and Gsdme-knockdown mice (FIG. 9C) under various light intensities. Knockdown of Gsdme markedly preserved STR amplitudes compared to controls.

FIGS. 10A-10B. Quantification of scotopic ERG parameters after Gsdme knockdown. Averaged amplitudes of positive STR (pSTR; FIG. 10A) and negative STR (nSTR; FIG. 10B) were analyzed across multiple light intensities. Gsdme knockdown significantly enhanced both pSTR and nSTR compared to scrambled controls.

FIGS. 11A-11C. Pharmacological inhibition of GSDME activation by 2-bromopalmitate (2-BP) attenuates optic nerve crush (ONC)-induced retinal ganglion cell (RGC) loss. (FIG. 11A) Experimental design illustrating flat-mounted retina segmentation and RGC counting. (FIG. 11B) Representative retinal flat-mounts immunostained for Brn3a showing RGC survival 14 days after ONC. (FIG. 11C) Quantification of Brn3a⁺ RGC density revealed significant protection in 2-BP-treated eyes compared with vehicle controls.

### DETAILED DESCRIPTION

Before the present disclosure is described in greater detail, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Although example embodiments of the present disclosure are explained in some instances in detail herein, it is to be understood that other embodiments are contemplated. Accordingly, it is not intended that the present disclosure be limited in its scope to the details of construction and arrangement of components set forth in the following description or illustrated in the drawings. The present disclosure is capable of other embodiments and of being practiced or carried out in various ways.

Where a range of values is provided, it is understood that each intervening value, to the tenth of the unit of the lower limit (unless the context clearly dictates otherwise), between the upper and lower limit of that range, and any other stated or intervening value in that stated range, is encompassed within the disclosure. The upper and lower limits of these smaller ranges may independently be included in the smaller ranges and are also encompassed within the disclosure, subject to any specifically excluded limit in the stated range. Where the stated range includes one or both of the limits, ranges excluding either or both of those included limits are also included in the disclosure.

As will be apparent to those of skill in the art upon reading this disclosure, each of the individual embodiments described and illustrated herein has discrete components and features which may be readily separated from or combined with the features of any of the other several embodiments without departing from the scope or spirit of the present disclosure. Any recited method can be carried out in the order of events recited or in any other order that is logically possible.

Embodiments of the present disclosure will employ, unless otherwise indicated, techniques of genetics, microbiology, biochemistry, molecular biology, cellular biology, tissue culture, therapeutic administrations and the like.

Before the embodiments of the present disclosure are described in detail, it is to be understood that, unless otherwise indicated, the present disclosure is not limited to particular materials, reagents, reaction materials, manufacturing processes, or the like, as such can vary. It is also to be understood that the terminology used herein is for purposes of describing particular embodiments only and is not intended to be limiting. It is also possible in the present disclosure that steps can be executed in different sequence where this is logically possible.

### Definitions

Unless otherwise defined, all technical and scientific terms used herein have the same meaning as commonly understood by one of ordinary skill in the art. Although methods and materials similar or equivalent to those described herein can be used in the practice or testing of the present disclosure, suitable methods and materials are described herein.

As used in the specification and the appended claims, the singular forms “a,” “an,” and “the” may include plural referents unless the context clearly dictates otherwise. Thus, for example, reference to “a support” includes a plurality of supports. In this specification and in the claims that follow, reference will be made to a number of terms that shall be defined to have the following meanings unless a contrary intention is apparent.

Unless otherwise indicated, all numbers expressing quantities of ingredients, properties such as reaction conditions, and so forth used in the specification and claims are to be understood as being modified in all instances by the term “about.” Accordingly, unless indicated to the contrary, the numerical parameters set forth in this specification and claims are approximations that can vary depending upon the desired properties sought to be obtained by the presently disclosed subject-matter.

The term “about”, when used herein in reference to a value, refers to a value that is similar, in context to the referenced value. In general, those skilled in the art, familiar with the context, will appreciate the relevant degree of variance encompassed by “about” in that context, for example, ±5%, 4%, 3%, 2%, etc.

Two events or entities are “associated” with one another, as that term is used herein, if the presence, level and/or form of one is correlated with that of the other. For example, a particular entity (e.g., polypeptide, genetic signature, metabolite, microbe, etc.) is considered to be associated with a particular disease, disorder, or condition, if its presence, level and/or form correlates with incidence of and/or susceptibility to the disease, disorder, or condition (e.g., across a relevant population). In some embodiments, two or more entities are physically “associated” with one another if they interact, directly or indirectly, so that they are and/or remain in physical proximity with one another. In some embodiments, two or more entities that are physically associated with one another are covalently linked to one another; in some embodiments, two or more entities that are physically associated with one another are not covalently linked to one another but are non-covalently associated, for example by means of hydrogen bonds, van der Waals interaction, hydrophobic interactions, magnetism, and combinations thereof.

As used herein, the term “comparable” refers to two or more agents, entities, situations, sets of conditions, etc., that may not be identical to one another but that are sufficiently similar to permit comparison there between so that one skilled in the art will appreciate that conclusions can reasonably be drawn based on differences or similarities observed. In some embodiments, comparable sets of conditions, circumstances, individuals, or populations are characterized by a plurality of substantially identical features and one or a small number of varied features. Those of ordinary skill in the art will understand, in context, what degree of identity is required in any given circumstance for two or more such agents, entities, situations, sets of conditions, etc. to be considered comparable. For example, those of ordinary skill in the art will appreciate that sets of circumstances, individuals, or populations are comparable to one another when characterized by a sufficient number and type of substantially identical features to warrant a reasonable conclusion that differences in results obtained or phenomena observed under or with different sets of circumstances, individuals, or populations are caused by or indicative of the variation in those features that are varied.

Those skilled in the art will appreciate that the term “composition”, as used herein, can be used to refer to a discrete physical entity that comprises one or more specified components. In general, unless otherwise specified, a composition can be of any suitable form—e.g., gel, liquid, solid, etc.

A composition or method described herein as “comprising” one or more named elements or steps is open-ended, meaning that the named elements or steps are essential to a particular aspect or embodiment, but other elements or steps can be added within the scope of the composition or method. To avoid prolixity, it is also understood that any composition or method described as “comprising” (or which “comprises”) one or more named elements or steps also describes the corresponding, more limited composition or method “consisting essentially of” (or which “consists essentially of”) the same named elements or steps, meaning that the composition or method includes the named essential elements or steps and can also include additional elements or steps that do not materially affect the basic and novel characteristic(s) of the composition or method. It is also understood that any composition or method described herein as “comprising” or “consisting essentially of” one or more named elements or steps also describes the corresponding, more limited, and closed-ended composition or method “consisting of” (or “consists of”) the named elements or steps to the exclusion of any other unnamed element or step. In any composition or method disclosed herein, known or disclosed equivalents of any named essential element or step can be substituted for that element or step.

In this disclosure, “consisting essentially of” or “consists essentially” or the like, when applied to methods and compositions encompassed by the present disclosure refers to compositions like those disclosed herein, but which may contain additional structural groups, composition components or method steps (or analogs or derivatives thereof as discussed above). Such additional structural groups, composition components or method steps, etc., however, do not materially affect the basic and novel characteristic(s) of the compositions or methods, compared to those of the corresponding compositions or methods disclosed herein. “Consisting essentially of” or “consists essentially” or the like, when applied to methods and compositions encompassed by the present disclosure have the meaning ascribed in U.S. Patent law and the term is open-ended, allowing for the presence of more than that which is recited so long as basic or novel characteristics of that which is recited is not changed by the presence of more than that which is recited, but excludes prior art embodiments.

As used herein, “Improved,” “increased” or “reduced,” or grammatically comparable comparative terms, indicate values that are relative to a baseline value or reference measurement. For example, in some embodiments, an assessed value achieved with an agent of interest may be “improved” relative to that obtained or expected in the absence of treatment or with a comparable reference agent or control. Alternatively, or additionally, in some embodiments, an assessed value achieved with an agent of interest may be “improved” relative to that obtained in the same subject or system under different conditions (e.g., prior to or after an event such as administration of an agent of interest), or in a different, comparable subject (e.g., in a comparable subject or system that differs from the subject or system of interest). In some embodiments, comparative terms refer to statistically relevant differences (e.g., that are of a prevalence and/or magnitude sufficient to achieve statistical relevance). Those skilled in the art will be aware, or will readily be able to determine, in a given context, a degree and/or prevalence of difference that is required or sufficient to achieve such statistical significance.

As used herein, “isolated” means separated from constituents that otherwise may be present, for example, separated from bacterial stains or species that are not desired, or separating from other constituents that may be present with the micro-organisms in nature.

As used herein, the term “encode” refers to principle that DNA can be transcribed into RNA, which can then be translated into amino acid sequences that can form proteins

As used herein, the terms “optional” or “optionally” means that the subsequently described event or circumstance can or cannot occur, and that the description includes instances where said event or circumstance occurs and instances where it does not.

As used herein, “individual”, “organism”, “host”, “subject”, and “patient” refers to any living entity comprised of at least one cell. A living organism can be as simple as, for example, a single isolated eukaryotic cell or cultured cell or cell line, or as complex as a mammal, including a human being, and animals (e.g., vertebrates, amphibians, fish, mammals, e.g., cats, dogs, horses, pigs, cows, sheep, rodents, rabbits, squirrels, bears, primates (e.g., chimpanzees, gorillas, and humans). These terms (“individual,” “subject,” “host,” and “patient,” used interchangeably herein also refer to any mammalian subject for whom diagnosis, treatment, or therapy is desired, particularly humans. In embodiments, subject may relate to particular components of the subject, for instance specific tissues or fluids of a subject (e.g., human tissue in a particular area of the body of a living subject), which may be in a particular location of the subject, referred to herein as an “area of interest” or a “region of interest.”

As used herein, “kit” means a collection of at least two components constituting the kit. Together, the components constitute a functional unit for a given purpose. Individual member components may be physically packaged together or separately. For example, a kit comprising an instruction for using the kit may or may not physically include the instruction with other individual member components. Instead, the instruction can be supplied as a separate member component, either in a paper form or an electronic form which may be supplied on computer readable memory device or downloaded from an internet website, or as recorded presentation.

As used herein, “instruction(s)” means documents describing relevant materials or methodologies pertaining to a kit. These materials may include any combination of the following: background information, list of components and their availability information (purchase information, etc.), brief or detailed protocols for using the kit, trouble-shooting, references, technical support, and any other related documents. Instructions can be supplied with the kit or as a separate member component, either as a paper form or an electronic form which may be supplied on computer readable memory device or downloaded from an internet website, or as recorded presentation. Instructions can comprise one or multiple documents and are meant to include future updates.

Reference throughout this specification to “one embodiment”, “an embodiment”, “another embodiment”, “some embodiment,” means that a particular feature, structure or characteristic described in connection with the embodiment is included in at least one embodiment of the present invention. Thus, appearances of the phrases “in one embodiment,” “in an embodiment,” “in another embodiment”, or “in some embodiment” in various places throughout this specification are not necessarily all referring to the same embodiment, but they may. Furthermore, the particular features, structures or characteristics may be combined in any suitable manner, as would be apparent to a person skilled in the art from this disclosure, in one or more embodiments. Furthermore, while some embodiments described herein include some, but not other, features included in other embodiments, combinations of features of different embodiments are meant to be within the scope of the invention. For example, in the appended claims, any of the claimed embodiments can be used in any combination.

A “control” sample or value refers to a sample that serves as a reference, usually a known reference, for comparison to a test sample or condition. For example, a test sample can include cells exposed to a test condition or a test agent, while the control is not exposed to the test condition or agent (e.g., negative control). The control can also be a positive control, e.g., a known primary cell or a cell exposed to known conditions or agents, for the sake of comparison to the test condition. A control can also represent an average value gathered from a plurality of samples, e.g., to obtain an average value. For therapeutic applications, a sample obtained from a patient suspected of having a given disorder or deficiency can be compared to samples from a known normal (non-deficient) individual. A control can also represent an average value gathered from a population of similar individuals, e.g., patient having a given deficiency or healthy individuals with a similar medical background, same age, weight, etc. A control value can also be obtained from the same individual, e.g., from an earlier-obtained sample, prior to the disorder or deficiency, or prior to treatment. One of skill will recognize that controls can be designed for assessment of any number of parameters.

The term “biological sample” encompasses a variety of sample types obtained from an organism or a cell line. The term encompasses blood and other liquid samples of biological origin, solid tissue samples, such as a biopsy specimen or tissue cultures or cells derived therefrom and the progeny thereof. The term includes samples that have been manipulated in any way after their procurement, such as by treatment with reagents, solubilization, or enrichment for certain components. The term includes a clinical sample, and includes cells in cell culture, cell supernatants, cell lysates, serum, plasma, biological fluids, and tissue samples.

The term “clinical well-being” as used herein, refers to a state or degree of clinical or physiological wellness or health of a patient. A clinician can evaluate a patient's clinical well-being by physical examination or performing one or more tests or assays.

“Inhibitors,” “activators,” and “modulators” of expression or of activity are used to refer to inhibitory, activating, or modulating molecules, respectively, identified using in vitro and in vivo assays for expression or activity of a described target protein (or encoding polynucleotide), e.g., ligands, agonists, antagonists, and their homologs and mimetics. The term “modulator” includes inhibitors and activators. Inhibitors are agents that, e.g., inhibit expression or bind to, partially or totally block stimulation or protease inhibitor activity, decrease, prevent, delay activation, inactivate, desensitize, or down regulate the activity of the described target protein, e.g., antagonists. Activators are agents that, e.g., induce or activate the expression of a described target protein or bind to, stimulate, increase, open, activate, facilitate, enhance activation or protease inhibitor activity, sensitize or up regulate the activity of described target protein (or encoding polynucleotide), e.g., agonists. Modulators include naturally occurring and synthetic ligands, antagonists and agonists (e.g., small chemical molecules, antibodies and the like that function as either agonists or antagonists). Such assays for inhibitors and activators include, e.g., applying putative modulator compounds to cells expressing the described target protein and then determining the functional effects on the described target protein activity, as described above. Samples or assays comprising described target protein that are treated with a potential activator, inhibitor, or modulator are compared to control samples without the inhibitor, activator, or modulator to examine the extent of effect. Control samples (untreated with modulators) are assigned a relative activity value of 100%. Inhibition of a described target protein is achieved when the activity value relative to the control is about 80%, optionally 50% or 25, 10%, 5% or 1%. Activation of the described target protein is achieved when the activity value relative to the control is 110%, optionally 150%, optionally 200, 300%, 400%, 500%, or 1000-3000% or higher.

The terms “administering,” “delivering,” and “introducing,” can be used interchangeably to indicate the introduction of a therapeutic composition or agent (e.g., compositions comprising one or more bacterial species as described herein) into the body of a subject. The therapeutic composition or agent can be administered through any appropriate means that results in the delivery of at least a portion of the composition or agent to a desired location in the subject such that the composition or agent retains its therapeutic capability. Useful methods of delivering the GSDME therapeutics described herein include, but are not limited to, intravenous, subretinal delivery, subconjunctival delivery, intracameral delivery, episcleral implants, topical administration, intravenous delivery, subcutaneous delivery, intradermal delivery, intracoronary delivery, intracardiac delivery, oral delivery, or any combination thereof.

The term “administered continuously” refers to the continuous delivery of a therapeutic agent, e.g., compound, molecule, peptide, biologic, chemical, etc. over a 24-hour period.

The term “therapeutically effective amount” refers to an amount of therapeutic agent effective to treat at least one symptom of a disease or disorder in a subject. In other words, such an amount is sufficient to bring about a beneficial or desired clinical effect. The “therapeutically effective amount” of the agent for administration may vary based upon the desired activity, the diseased state of the subject being treated, the dosage form, method of administration, subject factors such as the subject's sex, genotype, weight and age, the underlying causes of the condition or disease to be treated, the route of administration and bioavailability, the persistence of the administered agent in the body, evidence of natriuresis and/or diuresis, the type of formulation, and the potency of the agent.

As used herein, the terms “pharmaceutically acceptable” or “pharmacologically acceptable” refer to compositions that do not substantially produce adverse reactions, e.g., toxic, allergic, or immunological reactions, when administered to a subject.

The terms “therapy,” “treatment,” and “amelioration” refer to any reduction in the severity of symptoms, e.g., of a neurodegenerative disorder or neuronal injury. As used herein, the terms “treat” and “prevent” are not intended to be absolute terms. Treatment can refer to any delay in onset, amelioration of symptoms, improvement in patient survival, improved cognitive function or coordination, increase in survival time or rate, etc. The effect of treatment can be compared to an individual or pool of individuals not receiving the treatment, or to the same patient prior to treatment or at a different time during treatment. In some aspects, the severity of disease is reduced by at least 10%, as compared, e.g., to the individual before administration or to a control individual not undergoing treatment. In some aspects the severity of disease is reduced by at least 25%, 50%, 75%, 80%, or 90%, or in some cases, no longer detectable using standard diagnostic techniques. The terms “treating” or “treatment” as used herein refers to an alleviation of symptoms associated with a disorder or disease, or inhibition of further progression or worsening of those symptoms, or prevention or prophylaxis of the disease or disorder, or curing the disease or disorder. Similarly, as used herein, an “effective amount” or a “therapeutically effective amount” of a compound of the invention refers to an amount of the compound that alleviates, in whole or in part, symptoms associated with the disorder or condition, or halts or slows further progression or worsening of those symptoms or prevents or provides prophylaxis for the disorder or condition

As used throughout, the terms “nucleic acid,” “nucleic acid sequence,” “oligonucleotide,” “nucleotides,” or other grammatical equivalents as used herein mean at least two nucleotides, either deoxyribonucleotides or ribonucleotides, or analogs thereof, covalently linked together. Polynucleotides are polymers of any length, including, e.g., 20, 50, 100, 200, 300, 500, 1000, 2000, 3000, 5000, 7000, 10,000, etc. A polynucleotide described herein generally contains phosphodiester bonds, although in some cases, nucleic acid analogs are included that may have at least one different linkage, e.g., phosphoramidate, phosphorothioate, phosphorodithioate, or O-methylphophoroamidite linkages, and peptide nucleic acid backbones and linkages. Mixtures of naturally occurring polynucleotides and analogs can be made; alternatively, mixtures of different polynucleotide analogs, and mixtures of naturally occurring polynucleotides and analogs may be made. The following are non-limiting examples of polynucleotides: a gene or gene fragment, exons, introns, messenger RNA (mRNA), transfer RNA, ribosomal RNA, ribozymes, cDNA, cRNA, shRNA, siRNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component. The term also includes both double- and single-stranded molecules. Unless otherwise specified or required, the term polynucleotide encompasses both the double-stranded form and each of two complementary single-stranded forms known or predicted to make up the double-stranded form. A polynucleotide is composed of a specific sequence of four nucleotide bases: adenine (A), cytosine (C), guanine (G), thymine (T), and uracil (U) for thymine when the polynucleotide is RNA. Thus, the term “polynucleotide sequence” is the alphabetical representation of a polynucleotide molecule. Unless otherwise indicated, a particular polynucleotide sequence also implicitly encompasses conservatively modified variants thereof (e.g., degenerate codon substitutions) and complementary sequences as well as the sequence explicitly indicated. Specifically, degenerate codon substitutions may be achieved by generating sequences in which the third position of one or more selected (or all) codons is substituted with mixed-base and/or deoxy inosine residues.

Unless otherwise indicated, a particular nucleic acid sequence also implicitly encompasses conservatively modified variants thereof, alleles, orthologs, SNPs, and complementary sequences as well as the sequence explicitly indicated.

As used herein, “cDNA” refers to a DNA sequence that is complementary to an RNA transcript in a cell. It is a man-made molecule. Typically, cDNA is made in vitro by an enzyme called reverse-transcriptase using RNA transcripts as templates.

As used herein with reference to the relationship between DNA, cDNA, cRNA, RNA, protein/peptides, and the like “corresponding to” or “encoding” (used interchangeably herein) refers to the underlying biological relationship between these different molecules. As such, one of skill in the art would understand that operatively “corresponding to” can direct them to determine the possible underlying and/or resulting sequences of other molecules given the sequence of any other molecule which has a similar biological relationship with these molecules. For example, from a DNA sequence an RNA sequence can be determined and from an RNA sequence a cDNA sequence can be determined.

As used herein, “gene” can refer to a hereditary unit corresponding to a sequence of DNA that occupies a specific location on a chromosome and that contains the genetic instruction for a characteristic(s) or trait(s) in an organism. The term gene can refer to translated and/or untranslated regions of a genome. “Gene” can refer to the specific sequence of DNA that is transcribed into an RNA transcript that can be translated into a polypeptide or be a catalytic RNA molecule, including but not limited to, tRNA, siRNA, piRNA, miRNA, long-non-coding RNA and shRNA.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

Unless otherwise indicated, a particular nucleic acid sequence also implicitly encompasses conservatively modified variants thereof, alleles, orthologs, SNPs, and complementary sequences as well as the sequence explicitly indicated.

The terms “transfection”, “transduction”, “transfecting” or “transducing” can be used interchangeably and are defined as a process of introducing a nucleic acid molecule or a protein to a cell. Nucleic acids are introduced to a cell using non-viral or viral-based methods. The nucleic acid molecules may be gene sequences encoding complete proteins or functional portions thereof. Non-viral methods of transfection include any appropriate transfection method that does not use viral DNA or viral particles as a delivery system to introduce the nucleic acid molecule into the cell. Exemplary non-viral transfection methods include calcium phosphate transfection, liposomal transfection, nucleofection, sonoporation, transfection through heat shock, magnetifection and electroporation. In some embodiments, the nucleic acid molecules are introduced into a cell using electroporation following standard procedures well known in the art. For viral-based methods of transfection any useful viral vector may be used in the methods described herein. Examples for viral vectors include, but are not limited to retroviral, adenoviral, lentiviral and adeno-associated viral vectors. In some embodiments, the nucleic acid molecules are introduced into a cell using a retroviral vector following standard procedures well known in the art. The terms “transfection” or “transduction” also refer to introducing proteins into a cell from the external environment. Typically, transduction or transfection of a protein relies on attachment of a peptide or protein capable of crossing the cell membrane to the protein of interest. See, e.g., Ford et al. (2001) Gene Therapy 8:1-4 and Prochiantz (2007) Nat. Methods 4:119-20.

The word “expression” or “expressed” as used herein in reference to a gene means the transcriptional and/or translational product of that gene. The level of expression of a DNA molecule in a cell may be determined on the basis of either the amount of corresponding mRNA that is present within the cell or the amount of protein encoded by that DNA produced by the cell (Sambrook et al., 1989 Molecular Cloning: A Laboratory Manual, 18.1-18.88).

Expression of a transfected gene can occur transiently or stably in a cell. During “transient expression” the transfected gene is not transferred to the daughter cell during cell division. Since its expression is restricted to the transfected cell, expression of the gene is lost over time. In contrast, stable expression of a transfected gene can occur when the gene is co-transfected with another gene that confers a selection advantage to the transfected cell. Such a selection advantage may be a resistance towards a certain toxin that is presented to the cell. Expression of a transfected gene can further be accomplished by transposon-mediated insertion into to the host genome. During transposon-mediated insertion, the gene is positioned in a predictable manner between two transposon linker sequences that allow insertion into the host genome as well as subsequent excision.

The term “plasmid” refers to a nucleic acid molecule that encodes for genes and/or regulatory elements necessary for the expression of genes. Expression of a gene from a plasmid can occur in cis or in trans. If a gene is expressed in cis, gene and regulatory elements are encoded by the same plasmid. Expression in trans refers to the instance where the gene and the regulatory elements are encoded by separate plasmids.

The term “episomal” refers to the extra-chromosomal state of a plasmid in a cell. Episomal plasmids are nucleic acid molecules that are not part of the chromosomal DNA and replicate independently thereof.

The term “exogenous” refers to a molecule or substance (e.g., nucleic acid or protein) that originates from outside a given cell or organism. Conversely, the term “endogenous” refers to a molecule or substance that is native to, or originates within, a given cell or organism.

The term “vector” refers to a carrier DNA molecule into which a DNA sequence can be inserted for introduction into a host cell. In some embodiments, vectors of use in the invention are those capable of autonomous replication and/or expression of nucleic acids to which they are linked. Vectors capable of directing the expression of genes to which they are operatively linked are referred to herein as “expression vectors”. Thus, an “expression vector” is a specialized vector that contains the necessary regulatory regions needed for expression of a gene of interest in a host cell. In some embodiments the gene of interest is operably linked to another sequence in the vector, e.g., a promoter. Vectors include non-viral vectors such as plasmids and viral vectors.

A “viral vector” is a viral-derived nucleic acid that is capable of transporting another nucleic acid into a cell. A viral vector is capable of directing expression of a protein or proteins encoded by one or more genes carried by the vector when it is present in the appropriate environment. Examples for viral vectors include, but are not limited to retroviral, adenoviral, lentiviral and adeno-associated viral vectors.

The term “operably linked” refers to a functional linkage between a first nucleic acid sequence and a second nucleic acid sequence, such that the first and second nucleic acid sequences are transcribed into a single nucleic acid sequence. Operably linked nucleic acid sequences need not be physically adjacent to each other. The term “operably linked” also refers to a functional linkage between a nucleic acid expression control sequence (such as a promoter, or array of transcription factor binding sites) and a transcribable nucleic acid sequence, wherein the expression control sequence directs transcription of the nucleic acid corresponding to the transcribable sequence.

The terms “regulatory sequence” and “promoter” are used interchangeably herein, and refer to nucleic acid sequences, such as initiation signals, enhancers, and promoters, which induce or control transcription of protein coding sequences with which they are operatively linked. In some examples, transcription of a recombinant gene is under the control of a promoter sequence (or other transcriptional regulatory sequence) which controls the expression of the recombinant gene in a cell-type in which expression is intended. It will also be understood that the recombinant gene can be under the control of transcriptional regulatory sequences which are the same or which are different from those sequences which control transcription of the naturally-occurring form of a protein. In some instances the promoter sequence is recognized by the synthetic machinery of the cell, or introduced synthetic machinery, required for initiating transcription of a specific gene.

“Expression cassette” refers to a polynucleotide comprising a promoter or other regulatory sequence operably linked to a sequence encoding a protein.

The term “siRNA” refers to a nucleic acid that forms a double stranded RNA, which double stranded RNA has the ability to reduce or inhibit expression of a gene or target gene when the siRNA expressed in the same cell as the gene or target gene. In the context of this invention, the term “siRNA” includes miRNA. “siRNA” thus refers to the double stranded RNA formed by the complementary strands. The complementary portions of the siRNA that hybridize to form the double stranded molecule typically have substantial or complete identity. In one embodiment, an siRNA refers to a nucleic acid that has substantial or complete identity to a target gene and forms a double stranded siRNA. The sequence of the siRNA can correspond to the full length target gene, or a subsequence thereof. Typically, the siRNA is at least about 15-50 nucleotides in length (e.g., each complementary sequence of the double stranded siRNA is 15-50 nucleotides in length, and the double stranded siRNA is about 15-50 base pairs in length, preferable about preferably about 20-30 base nucleotides, preferably about 20-25 nucleotides in length, e.g., 20, 21, 22, 23, 24, 25, 26, 27, 28, 29, or 30 nucleotides in length.

The term “shRNA” refers generally to an siRNA that is introduced into a cell as part of a larger DNA construct. Typically, such constructs allow stable expression of the siRNA in cells after introduction, e.g., by integration of the construct into the host genome.

An “antisense” oligonucleotide or polynucleotide is a nucleotide sequence that is substantially complementary to a target polynucleotide or a portion thereof and has the ability to specifically hybridize to the target polynucleotide.

Ribozymes are enzymatic RNA molecules capable of catalyzing specific cleavage of RNA. The composition of ribozyme molecules preferably includes one or more sequences complementary to a target mRNA, and the well-known catalytic sequence responsible for mRNA cleavage or a functionally equivalent sequence (see, e.g., U.S. Pat. No. 5,093,246, which is incorporated herein by reference in its entirety). Ribozyme molecules designed to catalytically cleave target mRNA transcripts can also be used to prevent translation of subject target mRNAs.

The terms “polypeptide” and “peptide” are used interchangeably herein to refer to a polymer of amino acid residues in a single chain. The terms apply to amino acid polymers in which one or more amino acid residue is an artificial chemical mimetic of a corresponding naturally occurring amino acid, as well as to naturally occurring amino acid polymers and non-naturally occurring amino acid polymers. Amino acid polymers may comprise entirely L-amino acids, entirely D-amino acids, or a mixture of L- and D-amino acids. The term “protein” as used herein refers to either a polypeptide or a dimer (i.e., two) or multimer (i.e., three or more) of single chain polypeptides. The single chain polypeptides of a protein may be joined by a covalent bond, e.g., a disulfide bond, or non-covalent interactions. The terms “portion” and “fragment” are used interchangeably herein to refer to parts of a polypeptide, nucleic acid, or other molecular construct.

The term “amino acid” refers to naturally occurring and synthetic amino acids, as well as amino acid analogs and amino acid mimetics that function in a manner similar to the naturally occurring amino acids. Naturally occurring amino acids are those encoded by the genetic code, as well as those amino acids that are later modified, e.g., hydroxyproline, y-carboxyglutamate, and O-phosphoserine. Amino acid analogs refer to compounds that have the same basic chemical structure as a naturally occurring amino acid, i.e., an a carbon that is bound to a hydrogen, a carboxyl group, an amino group, and an R group, e.g., homoserine, norleucine, methionine sulfoxide, methionine methyl sulfonium. Such analogs have modified R groups (e.g., norleucine) or modified peptide backbones, but retain the same basic chemical structure as a naturally occurring amino acid. Amino acid mimetics refers to chemical compounds that have a structure that is different from the general chemical structure of an amino acid, but that functions in a manner similar to a naturally occurring amino acid.

Amino acids may be referred to herein by either their commonly known three letter symbols or by the one-letter symbols recommended by the IUPAC-IUB Biochemical Nomenclature Commission. Nucleotides, likewise, may be referred to by their commonly accepted single-letter codes.

The amino acids in the polypeptides described herein can be any of the 20 naturally occurring amino acids, D-stereoisomers of the naturally occurring amino acids, unnatural amino acids and chemically modified amino acids. Unnatural amino acids (that is, those that are not naturally found in proteins) are also known in the art, as set forth in, for example, Zhang et al. “Protein engineering with unnatural amino acids,” Curr. Opin. Struct. Biol. 23(4): 581-87 (2013); Xie et al. “Adding amino acids to the genetic repertoire,” Curr. Opin. Chem. Biol. 9(6): 548-54 (2005); and all references cited therein. Beta and gamma amino acids are known in the art and are also contemplated herein as unnatural amino acids.

In accordance with standard nomenclature, amino acid residue sequences are denominated by either a three letter or a single letter code as indicated as follows, for example: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (lie, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V). “Protein” and “Polypeptide” can refer to a molecule composed of one or more chains of amino acids in a specific order. The term protein is used interchangeable with “polypeptide.” The order is determined by the base sequence of nucleotides in the gene coding for the protein. Proteins can be involved in the structure, function, and regulation of various functions.

The term “identity” or “substantial identity,” as used in the context of a polynucleotide or polypeptide sequence described herein, refers to a sequence that has at least 60% sequence identity to a reference sequence. Alternatively, percent identity can be any integer from 60% to 100%. Exemplary embodiments include at least: 60%, 65%, 70%, 75%, 80%, 85%, 88%, 90%, 91%, 92%, 93%, 94%, 95%, 96%, 97%, 98%, or 99%, as compared to a reference sequence using the programs described herein; preferably BLAST using standard parameters, as described below. One of skill will recognize that these values can be appropriately adjusted to determine corresponding identity of proteins encoded by two nucleotide sequences by taking into account codon degeneracy, amino acid similarity, reading frame positioning and the like.

For sequence comparison, typically one sequence acts as a reference sequence to which test sequences are compared. When using a sequence comparison algorithm, test and reference sequences are entered into a computer, subsequence coordinates are designated, if necessary, and sequence algorithm program parameters are designated. Default program parameters can be used, or alternative parameters can be designated. The sequence comparison algorithm then calculates the percent sequence identities for the test sequences relative to the reference sequence, based on the program parameters.

A “comparison window,” as used herein, includes reference to a segment of any one of the number of contiguous positions selected from the group consisting of from 20 to 600, usually about 50 to about 200, more usually about 100 to about 150 in which a sequence may be compared to a reference sequence of the same number of contiguous positions after the two sequences are optimally aligned. Methods of alignment of sequences for comparison are well-known in the art. Optimal alignment of sequences for comparison may be conducted by the local homology algorithm of Smith & Waterman Add. APL. Math. 2:482 (1981), by the homology alignment algorithm of Needleman & Wunsch J. Mol. Biol. 48:443 (1970), by the search for similarity method of Pearson & Lipman Proc. Natl. Acad. Sci. (U.S.A.) 85: 2444 (1988), by computerized implementations of these algorithms (e.g., BLAST), or by manual alignment and visual inspection.

Algorithms that are suitable for determining percent sequence identity and sequence similarity are the BLAST and BLAST 2.0 algorithms, which are described in Altschul et al. (1990) J. Mol. Biol. 215: 403-10 and Altschul et al. (1977) Nucleic Acids Res. 25: 3389-402, respectively. Software for performing BLAST analyses is publicly available through the National Center for Biotechnology Information (NCBI) web site. The algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul et al. (1977)). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a word size (W) of 28, an expectation (E) of 10, M=1, N=−2, and a comparison of both strands. For amino acid sequences, the BLASTP program uses as defaults a word size (W) of 3, an expectation (E) of 10, and the BLOSUM62 scoring matrix (see Henikoff & Henikoff, Proc. Natl. Acad. Sci. USA 89:10915 (1989)).

The BLAST algorithm also performs a statistical analysis of the similarity between two sequences (see, e.g., Karlin & Altschul, Proc. Nat'l. Acad. Sci. USA 90:5873-5787 (1993)). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.01, more preferably less than about 10⁻⁵, and most preferably less than about 10⁻²⁰.

The terms “co-administration” or “co-administered” as used herein refer to the administration of at least two compounds or agent(s) or therapies to a subject. In some embodiments, the co-administration of two or more agents/therapies is concurrent. In other embodiments, a first agent/therapy is administered prior to a second agent/therapy in this aspect, each component may be administered separately, but sufficiently close in time to provide the desired effect, in particular a beneficial, additive, or synergistic effect. Those of skill in the art understand that the formulations and/or routes of administration of the various agents/therapies used may vary. The appropriate dosage for co-administration can be readily determined by one skilled in the art. In some embodiments, when agents/therapies are co-administered, the respective agents/therapies are administered at lower dosages than appropriate for their administration alone. Thus, co-administration is especially desirable in embodiments where the co-administration of the agents/therapies lowers the requisite dosage of a known potentially harmful (e.g., toxic) agent(s).

The term “composition” as used herein refers to a product comprising the specified ingredients in the specified amounts, as well as any product which results, directly or indirectly, from combination of the specified ingredients in the specified amounts. Such a term in relation to a pharmaceutical composition is intended to encompass a product comprising the active ingredient(s), and the inert ingredient(s) that make up the carrier, as well as any product which results, directly or indirectly, from combination, complexation, or aggregation of any two or more of the ingredients, or from dissociation of one or more of the ingredients, or from other types of reactions or interactions of one or more of the ingredients. Accordingly, the pharmaceutical compositions of the present disclosure encompass any composition made by admixing a compound of the present disclosure and a pharmaceutically acceptable carrier.

When a compound of the present disclosure is used contemporaneously with one or more other drugs, a pharmaceutical composition containing such other drugs in addition to the compound of the present disclosure is contemplated. Accordingly, the pharmaceutical compositions of the present disclosure include those that also contain one or more other active ingredients, in addition to a compound of the present disclosure. The weight ratio of the compound of the present disclosure to the second active ingredient may be varied and will depend upon the effective dose of each ingredient. Generally, an effective dose of each will be used. Thus, for example, but not intended to be limiting, when a compound of the present disclosure is combined with another agent, the weight ratio of the compound of the present disclosure to the other agent will generally range from about 1000:1 to about 1:1000, preferably about 200:1 to about 1:200. Combinations of a compound of the present disclosure and other active ingredients will generally also be within the aforementioned range, but in each case, an effective dose of each active ingredient should be used. In such combinations the compound of the present disclosure and other active agents may be administered separately or in conjunction. In addition, the administration of one element may be prior to, concurrent to, or subsequent to the administration of other agent(s).

A composition of the disclosure can be a liquid solution, suspension, emulsion or a powder. Various delivery systems are known and can be used to administer a composition of the disclosure, e.g. encapsulation in liposomes, microparticles, microcapsules, and the like.

Compositions for administration may include sterile aqueous or non-aqueous solvents, such as water, isotonic saline, isotonic glucose solution, buffer solution, or other solvents conveniently used for parenteral administration of therapeutically active agents, stabilizers, buffers, or preservatives, e.g. antioxidants such as methylhydroxybenzoate or similar additives.

A composition of the disclosure may be sterilized by, for example, addition of sterilizing agents to the composition, irradiation of the composition, or heating the composition. Alternatively, the compounds or compositions of the present disclosure may be provided as sterile solid preparations e.g. lyophilized powder, which are readily dissolved in sterile solvent immediately prior to use.

After pharmaceutical compositions have been prepared, they can be placed in an appropriate container and labeled for treatment of an indicated condition. For administration of a composition of the disclosure, such labeling would include amount, frequency, and method of administration.

The term “freeze-dried (lyophilized) as used herein refers to a preparation of bacterial cells that have been initially frozen and the water content removed by vacuum.

The term “pharmaceutically acceptable carrier” as used herein refers to a diluent, adjuvant, excipient, or vehicle with which a probe of the disclosure is administered and which is approved by a regulatory agency of the Federal or a state government or listed in the U.S. Pharmacopeia or other generally recognized pharmacopeia for use in animals, and more particularly in humans. Such pharmaceutical carriers can be liquids, such as water and oils, including those of petroleum, animal, vegetable or synthetic origin, such as peanut oil, soybean oil, mineral oil, sesame oil and the like. The pharmaceutical carriers can be saline, gum acacia, gelatin, starch paste, talc, keratin, colloidal silica, urea, and the like. When administered to a patient, the probe and pharmaceutically acceptable carriers can be sterile. Water is a useful carrier when the probe is administered intravenously. Saline solutions and aqueous dextrose and glycerol solutions can also be employed as liquid carriers, particularly for injectable solutions. Suitable pharmaceutical carriers also include excipients such as glucose, lactose, sucrose, glycerol monostearate, sodium chloride, glycerol, propylene, glycol, water, ethanol and the like. The present compositions, if desired, can also contain minor amounts of wetting or emulsifying agents, or pH buffering agents. The present compositions advantageously may take the form of solutions, emulsion, sustained-release formulations, or any other form suitable for use.

The term “preventing” means to stop or hinder a disease, disorder, or symptom of a disease or condition through some action.

The term “reducing” means to diminish in extent, amount, or degree.

The term “therapeutic agent” as used herein refers to a therapeutic substance selected from a group consisting of, but not limited to, analgesics, anesthetics, anti-inflammatory agents, antiasthma agents, antibiotics (including penicillins), anticoagulants, antihistamines, antitussives, antihypertensive agents, antimuscarinic agents, antimycobacterial agents, antioxidant agents, antipyretics, immunosuppressants, immunostimulants, antiviral agents, bacteriostatic agents, bronchodilators, buffering agents, contrast media, corticosteroids, cough suppressants (expectorants and mucolytics), diagnostic agents, diagnostic imaging agents, free radical scavenging agents, growth factors, haemostatics, immunological agents, lipid regulating agents, muscle relaxants, proteins, peptides and polypeptides, prostaglandins, radio-pharmaceuticals, time release binders, anti-allergic agents, stimulants and anoretics, steroids, sympathomimetics, vasodilators, and xanthines.

As used herein, “knockdown” refers to temporary halting or decrease the expression of one or more targeted genes.

As used throughout, the term “Cas9 polypeptide” means a Cas9 protein or a fragment thereof present in any bacterial species that encodes a Type II CRISPR/Cas9 system. See, for example, Makarova et al. Nature Reviews, Microbiology, 9: 467-477 (2011), including supplemental information, hereby incorporated by reference in its entirety. For example, the Cas9 protein or a fragment thereof can be from Streptococcus pyogenes. Full-length Cas9 is an endonuclease comprising a recognition domain and two nuclease domains (HNH and RuvC, respectively) that creates double-stranded breaks in DNA sequences. In the amino acid sequence of Cas9, HNH is linearly continuous, whereas RuvC is separated into three regions, one left of the recognition domain, and the other two right of the recognition domain flanking the HNH domain. Cas9 from Streptococcus pyogenes is targeted to a genomic site in a cell by interacting with a guide RNA that hybridizes to a 20-nucleotide DNA sequence that immediately precedes an NGG motif recognized by Cas9. This results in a double-strand break in the genomic DNA of the cell.

As used throughout, a dCas9 polypeptide is a deactivated or nuclease-dead Cas9 (dCas9) that has been modified to inactivate Cas9 nuclease activity. Modifications include, but are not limited to, altering one or more amino acids to inactivate the nuclease activity or the nuclease domain. For example, and not to be limiting, D10A and H840A mutations can be made in Cas9 from Streptococcus pyogenes to inactivate Cas9 nuclease activity. Other modifications include removing all or a portion of the nuclease domain of Cas9, such that the sequences exhibiting nuclease activity are absent from Cas9. Accordingly, a dCas9 may include polypeptide sequences modified to inactivate nuclease activity or removal of a polypeptide sequence or sequences to inactivate nuclease activity. The dCas9 retains the ability to bind to DNA even though the nuclease activity has been inactivated. Accordingly, dCas9 includes the polypeptide sequence or sequences required for DNA binding but includes modified nuclease sequences or lacks nuclease sequences responsible for nuclease activity. It is understood that similar modifications can be made to inactivate nuclease activity in other site-directed nucleases, for example in Cpf1 or C2c2.

In some examples, the dCas9 protein is a full-length Cas9 sequence from S. pyogenes lacking the polypeptide sequence of the RuvC nuclease domain and/or the HNH nuclease domain and retaining the DNA binding function. In other examples, the dCas9 protein sequences have at least 30%, 40%, 50%, 60%, 70%, 80%, 90%, 95%, 98% or 99% identity to Cas9 polypeptide sequences lacking the RuvC nuclease domain and/or the HNH nuclease domain and retains DNA binding function.

As used throughout, the term “Cas13 polypeptide” means a Cas13 protein or a fragment thereof present in any bacterial species that encodes a Type VI CRISPR/Cas13 system. Examples include dPspCas13b, dLwaCas13a, and dRfxCas13d. See, for example, Abudayyeh et al., Science. 2016 August 5; 353(6299): aaf5573. doi:10.1126/science.aaf5573, including supplemental information, hereby incorporated by reference in its entirety; Cox et al., Science 358, 1019-1027 (2017) including supplemental information, hereby incorporated by reference in its entirety; and Tang et al., Front. Cell Dev. Biol., 27 Jul. 2021 Sec. Epigenomics and Epigenetics Volume 9-2021 | https://doi.org/10.3389/fcell.2021.677587. For example, the Cas13 protein or a fragment thereof with ssRNA targeting activity can be from Leptotrichia wadei, Leptotrichia shahii, Prevotella sp. P5-125 (PspCas13b), or Ruminococcus flavefaciens. Generally, Cas13 enzymes have two higher eukaryotes and prokaryotes nucleotide-binding (HEPN) endoRNase domains that mediate precise RNA cleavage with a preference for targets with protospacer flanking sites (PFSs) observed biochemically and in bacteria.

As used throughout, a dCas13 polypeptide is a deactivated or nuclease-dead Cas13 (dCas13) that has been modified to inactivate Cas13 nuclease activity. Modifications include, but are not limited to, altering one or more amino acids to inactivate the nuclease activity or the nuclease domain. For example, and not to be limiting, H133A and H1058A mutations can be made in Cas13 HEPN domains from Prevotella sp. P5-125 (PspCas13b) to inactivate Cas13 nuclease activity (see, for example, Cox et al., Science 358, 1019-1027 (2017) including supplemental information, hereby incorporated by reference in its entirety, and International Patent Publication WO 2019/005884, also incorporated by reference in its entirety). Other modifications include removing all or a portion of the nuclease domain of Cas13 (for example, A984-1090 H133A of Cas13b is from Prevotella sp. P5-125; see, for example, Programmable m(6)A modification of cellular RNAs with a Cas13-directed methyltransferase. Wilson C, Chen P J, Miao Z, Liu D R. Nat Biotechnol. 2020 Jun. 29. pii: 10.1038/s41587-020-0572-6. doi: 10.1038/s41587-020-0572-6. 10.1038/s41587-020-0572-6 PubMed 32601430), such that the sequences exhibiting nuclease activity are absent from Cas13. Additional mutations can include R474A/R1046A in dCas13 from L. wadei and mutations R239R/H244A/ and R858A/H863A from Ruminococcus flavefaciens strain XPD3002. Accordingly, a dCas13 may include polypeptide sequences modified to inactivate nuclease activity or removal of a polypeptide sequence or sequences to inactivate nuclease activity. The dCas13 retains the ability to target ssRNA even though the nuclease activity has been inactivated. Accordingly, dCas13 includes the polypeptide sequence or sequences required for ssRNA targeting but includes modified nuclease sequences or lacks nuclease sequences responsible for nuclease activity.

In some examples, the dCas13 protein is a full-length Cas13 sequence from L. wadei, L. shahii, Prevotella sp. P5-125 (PspCas13b), or R. flavefaciens having one or more mutations in one or more HEPN domains and retaining the ssRNA targeting function. In other examples, the dCas13 protein sequences have at least 30%, 40%, 50%, 60%, 70%, 80%, 90%, 95%, 98% or 99% identity to Cas13 polypeptide sequences with HEPN mutations and retains RNA binding function.

### Discussion

Described herein are compositions, pharmaceutical compositions, methods, and kits relating to knocking-down, inhibiting, or otherwise reducing the activity and/or expression of Gasdermin E. Methods as described herein can be indicated, for example, in subjects having or suspected of having, for example, Traumatic Optic Neuropathy (TON), Glaucoma, juvenile glaucoma, Leber's Hereditary Optic Neuropathy, Optic Neuritis, Ischemic Optic Neuropathy, Diabetic Retinopathy, Retinal Occlusion, Anterior Ischemic Optic Neuropathy (AION), Infectious Neuropathies, Neuromyelitis optica (NMO), Behçet's disease, optic nerve hypoplasia, Central or branch retinal artery occlusion (CRAO, BRAO), Radiation Optic Neuropathy, Optic nerve glioma, Age-related macular degeneration (AMD).

### I. ANTI-GASDERMIN E (GSDME) THERAPEUTICS

Described herein are anti-Gasdermin E (GSDME) therapeutics that reduce the activity of, block, inhibit, knockdown, reduce expression, halt expression, and the like of Gasdermin E (for example, Human GSDME gene ID: 1687; Mouse GSDME gene ID: 54722). Therapeutics as described herein can comprise, for example, one or more nucleotides (for example, shRNA or siRNA) or small molecules.

### A. Nucleotides

Described herein are nucleotides (also referred to herein as polynucleotides) targeting Gasdermin E. Such polynucleotides include, for example and without intending to be limiting, anti-Gasdermin E short hairpin RNA (shRNA) and small interfering RNA (siRNA).

In embodiments, shRNA according to the present disclosure can comprise one or more of any of SEQ ID NOs: 1-19.

In embodiments, siRNA according to the present disclosure can comprise one or more of any of SEQ ID NOs: 20-29.

In each case, where a specific nucleotide sequence is recited, embodiments comprising a sequence having at least 75%, at least 80%, at least 85%, at least 90% (e.g. at least 90%, 91%, 92%, 93%, 94%, 95%, 96%, 97%, 98%, or 99%) identity to the recited sequence (e.g., SEQ ID NOs: 1-30) are also provided.

In each case, where a specific nucleotide sequence is recited, embodiments consisting essentially of a sequence having at least 75%, at least 80%, at least 85%, at least 90% (e.g. at least 90%, 91%, 92%, 93%, 94%, 95%, 96%, 97%, 98%, or 99%) identity to the recited sequence (e.g., SEQ ID NOs: 1-30) are also provided.

In each case, where a specific nucleotide sequence is recited, embodiments consisting of a sequence having at least 75%, at least 80%, at least 85%, at least 90% (e.g. at least 90%, 91%, 92%, 93%, 94%, 95%, 96%, 97%, 98%, or 99%) identity to the recited sequence (e.g., SEQ ID NOs: 1-30) are also provided.

Nucleotides as described herein can be delivered to cells or subjects as described herein by a number of methods, for example, non-viral and viral methodologies.

Exemplary non-viral transfection methods include, without intending to be limiting, calcium phosphate transfection, Iiposomal transfection, nucleofection, sonoporation, transfection through heat shock, magnetifection and electroporation.

Nucleotides as described herein can be utilized with or in conjunction with various viral vectors. Examples for viral vectors include, but are not limited to retroviral, adenoviral, lentiviral and adeno-associated viral vectors. In certain embodiments, AAV serotypes AAV1 and AAV2 are utilized to deliver nucleotides as described herein by viral transduction to a cell or a subject as described herein. A variety of promotors can be utilized in viral vectors according to the present disclosure, for example, from cell-type agnostic promoters such as the CMV or EF1a or U6 promoter, or cell-type specific promoters, for example and without intending to be limiting, an RGC-specific promoter such as that described in Hanlon K S, Chadderton N, Palfi A, Blanco Fernandez A, Humphries P, Kenna P F, Millington-Ward S, Farrar G J. A Novel Retinal Ganglion Cell Promoter for Utility in AAV Vectors. Front Neurosci. 2017 Sep. 21; 11:521. doi: 10.3389/fnins.2017.00521. PMID: 28983234; PMCID: PMC5613148., the contents of which are incorporated by reference for RGC-specific promoters. Additional RGC-specific promoters that can be utilized according to aspects of the present disclosure to drive expression of nucleotides described herein include, without intending to be limiting: y-Synuclein Promoter (nSncg), Nefh promoter, Thy1 promoter, Brn3b promoter, DCX promoter, Rbpms promoter, and/or a Syn promoter.

Additionally, components of the compositions, systems, and methods as described herein (for example, anti-Gasdermin E nucleotides can) be used in combination with clustered regularly interspaced short palindromic repeat inhibition (CRISPRi) tools. Described herein are compositions, methods, and kits of utilizing catalytically-dead CRISPR-associated (Cas) enzymes, for example, of RNA-targeting (also referred to herein as “RNA-guided”) type III (i.e., Csm/Csr), type VI (i.e. Cas13), or type II (i.e., Cas9) CRISPR-Cas systems to knockdown Gasdermin E expression or otherwise inhibit Gasdermin E activity. The utility of the system, to influence physiological outcomes can also be studied.

In some embodiments, the knock down of a nucleic acid sequence as described herein is performed using the CRIPSR/dCas9 system or CRIPSR/dCas13 system. The CRISPR/dCas9 system, for example, includes a dCas9 protein and at least one to two ribonucleic acids that are capable of directing the dCas9 protein to and hybridizing to a target motif in the genome that is to be replaced. These ribonucleic acids are commonly referred to as the “single guide RNA” or “sgRNA.”

In embodiments, a dCas9 or dCas13 protein used according to the present disclosure can be a naturally occurring dCas9 or dCas13 protein or a functional derivative thereof. A “functional derivative” of a native sequence polypeptide is a compound having a qualitative biological property in common with a native sequence polypeptide. “Functional derivatives” include, but are not limited to, fragments of a native sequence and derivatives of a native sequence polypeptide and its fragments, provided that they have a biological activity in common with a corresponding native sequence polypeptide. A biological activity contemplated herein is the ability of the functional derivative of dCas9 to localize a nucleotide to a target sequence. Suitable functional derivatives of a dCas9 or dCas13 polypeptide or a fragment thereof include but are not limited to mutants, fusions, covalent modifications of dCas9 protein or dCas13 protein or a fragment thereof. Examples of dCas9 and dCas13 proteins are discussed in the definitions above.

The sgRNAs can be selected depending on the particular CRISPR/Cas system employed and the sequence of the target polynucleotide. In some embodiments, the one to two ribonucleic acids are designed to hybridize to a target motif immediately adjacent to a deoxyribonucleic acid motif recognized by the Cas9 protein. In some embodiments, each of the one to two ribonucleic acids are designed to hybridize to target motifs immediately adjacent to deoxyribonucleic acid motifs recognized by the Cas9 protein, wherein the target motifs flank the genomic sequence to be replaced. Guide RNAs can be designed using software that is readily available, for example, at http://crispr.mit.edu. Examples of guide RNAs can be found in the Informal Sequence Listing below (SEQ ID NOs: 31-35 for murine GSDME and SEQ ID NOs: 36-40.

In some embodiments, the sgRNAs can also be selected to minimize hybridization with nucleic acid sequences other than the target polynucleotide sequence. In some embodiments, the one to two ribonucleic acids are designed to hybridize to a target motif that contains at least two mismatches when compared with all other genomic nucleotide sequences in the cell to minimize off-target effects of the CRISPR/Cas9 system. Those skilled in the art will appreciate that a variety of techniques can be used to select suitable target motifs for minimizing off-target effects (e.g., bioinformatics analyses). Methods of using the CRISPR/Cas9 system to reduce gene expression are described in various publications, e.g., US Pat. Pub. Nos. 2014/0170753 and 2016/0257974, the disclosures of which hereby are incorporated by reference in their entirety.

Those skilled in the art will readily appreciate how to use various approaches described herein to knock-down or otherwise inhibit Gasdermin E activity, e.g., siRNA, shRNA, small molecules, and CRISPRi systems.

### B. Small Molecules

Also described herein are small molecules targeting Gasdermin E. Small molecules are typically organic molecules less than 2500 with a carbon backbone that are not polynucleotides or polypeptides. Examples of small nucleotides that can be utilized as therapeutics as described herein include, without intending to be limiting, small molecules targeting or otherwise selective for Gasdermin E comprise any one or more of CuET (copper diethyldithiocarbamate), Disulfiram 20-hydroxyecdysone, Ac-DMPD-CMK, Ac-DMLD-CMK, Dimethyl Fumarate (DMF), or 2-bromopalmitate (2-BP), individually or in any combination of any thereof.

### II. PHARMACEUTICAL COMPOSITIONS AND FORMULATIONS

Compositions comprising one or more anti-Gasdermin therapeutics as discussed herein (for example, nucleotides and small molecules) and a pharmaceutically acceptable carrier are also provided. The compositions may further comprise a diluent, solubilizer, emulsifier, preservative, and/or adjuvant to be used with the methods disclosed herein.

In certain embodiments, acceptable formulation materials preferably are nontoxic to recipients at the dosages and concentrations employed. In certain embodiments, the formulation material(s) are for s.c. and/or I.V. administration. In certain embodiments, the pharmaceutical composition can contain formulation materials for modifying, maintaining or preserving, for example, the pH, osmolality, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates or other organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); fillers; monosaccharides, disaccharides, and other carbohydrates (such as glucose, mannose or dextrins); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (such as sucrose or sorbitol); tonicity enhancing agents (such as alkali metal halides, preferably sodium or potassium chloride, mannitol sorbitol); delivery vehicles; diluents; excipients and/or pharmaceutical adjuvants. (Allen (2012) Remington—The Science and Practice of Pharmacy, 22d Edition, Lloyd V, Allen, ed., The Pharmaceutical Press). In certain embodiments, the optimal pharmaceutical composition is determined by one skilled in the art depending upon, for example, the intended route of administration, delivery format and desired dosage. See, for example, Allen (2012) Remington—The Science and Practice of Pharmacy, 22d Edition, Lloyd V, Allen, ed., The Pharmaceutical Press.

In certain embodiments, the primary vehicle or carrier in a pharmaceutical composition can be either aqueous or non-aqueous in nature. For example, in certain embodiments, a suitable vehicle or carrier can be water for injection, physiological saline solution, possibly supplemented with other materials common in compositions for parenteral administration. In certain embodiments, the saline comprises isotonic phosphate-buffered saline. In certain embodiments, neutral buffered saline or saline mixed with serum albumin are further exemplary vehicles. In certain embodiments, pharmaceutical compositions comprise a pH controlling buffer such phosphate-buffered saline or acetate-buffered saline. In certain embodiments, a composition comprising micro-organisms described herein can be prepared for storage by mixing the selected composition having the desired degree of purity with optional formulation agents (see Allen (2012) Remington—The Science and Practice of Pharmacy, 22d Edition, Lloyd V, Allen, ed., The Pharmaceutical Press) in the form of a lyophilized cake or an aqueous solution. Further, in certain embodiments, a composition comprising micro-organisms described herein can be formulated as a lyophilizate using appropriate excipients. In some instances, appropriate excipients may include a cryo-preservative, a bulking agent, a surfactant, or a combination of any thereof. Exemplary excipients include one or more of a polyol, a disaccharide, or a polysaccharide, such as, for example, mannitol, sorbitol, sucrose, trehalose, and dextran 40. In some instances, the cryo-preservative may be sucrose or trehalose. In some instances, the bulking agent may be glycine or mannitol. In one example, the surfactant may be a polysorbate such as, for example, polysorbate-20 or polysorbate-80.

In certain embodiments, the pharmaceutical composition can be selected for parental delivery, in particular, intravitreal delivery, subconjunctival injection, and/or topical (eye drop) administration. The preparation of such pharmaceutically acceptable compositions is within the ability of one skilled in the art.

In certain embodiments, the formulation components are present in concentrations that are acceptable to the site of administration. In certain embodiments, buffers are used to maintain the composition at physiological pH or at a slightly lower pH, typically within a pH range of from about 5 to about 8. For example, the pH may be 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8. 6.9, 6.9, 7.0, 7.1, 7.2, 7.3, 7.4, 7.5, 7.6, 7.7, 7.8, 7.9, 8.0, 8.1, 8.2, 8.3, 8.4, or 8.5. In some instances, the pH of the pharmaceutical composition may be in the range of 6.6-8.5 such as, for example, 7.0-8.5, 6.6-7.2, 6.8-7.2, 6.8-7.4, 7.2-7.8, 7.0-7.5, 7.5-8.0, 7.2-8.2, 7.6-8.5, or 7.8-8.3. In some instances, the pH of the pharmaceutical composition may be in the range of 5.5-7.5 such as, for example, 5.5-5.8, 5.5-6.0, 5.7-6.2, 5.8-6.5, 6.0-6.5, 6.2-6.8, 6.5-7.0, 6.8-7.2, or 6.8-7.5. In some instances, the pH of the pharmaceutical composition may be in the range of 4.0-5.5 such as, for example, 4.0-4.3, 4.0-4.5, 4.2-4.8, 4.5-4.8, 4.5-5.0, 4.8-5.2, or 5.0-5.5. In an embodiment, the pH is 7.2.

In certain embodiments when parenteral administration is contemplated, a therapeutic composition can be in the form of a pyrogen-free, parenterally acceptable aqueous solution comprising micro-organisms described herein in a pharmaceutically acceptable vehicle. In certain embodiments, a vehicle for parenteral injection is sterile distilled water in which micro-organisms compositions described herein are formulated as a sterile, isotonic solution and properly preserved. In certain embodiments, the preparation can involve the formulation of the desired molecule with an agent, such as injectable microspheres, bio-erodible particles, polymeric compounds (such as polylactic acid or polyglycolic acid), beads or liposomes, that can provide for the controlled or sustained release of the product which can then be delivered via a depot injection. In certain embodiments, hyaluronic acid can also be used, and can have the effect of promoting sustained duration in the circulation. In certain embodiments, implantable drug delivery devices can be used to introduce the desired molecule.

In certain embodiments, it is contemplated that formulations can be administered orally. In certain embodiments, micro-organisms described herein that are administered in this fashion can be formulated with or without carriers customarily used in compounding solid dosage forms, such as tablets and capsules. In certain embodiments, a capsule can be designed to release the active portion of the formulation at the point in the gastrointestinal tract when bioavailability is maximized, and pre-systemic degradation is minimized. In certain embodiments, diluents, flavorings, low melting point waxes, vegetable oils, lubricants, suspending agents, tablet disintegrating agents, and binders can also be employed.

The pharmaceutical composition to be used for in vivo administration typically is sterile. In certain embodiments, sterilization is accomplished by filtration through sterile filtration membranes. In certain embodiments, where the composition is lyophilized, sterilization using this method can be conducted either prior to or following lyophilization and reconstitution. In certain embodiments, the composition for parenteral administration can be stored in lyophilized form or in a solution. In certain embodiments, parenteral compositions generally are placed into a container having a sterile access port, for example, an intravenous solution bag or vial having a stopper pierceable by a hypodermic injection needle.

In certain embodiments, once the pharmaceutical composition has been formulated, it can be stored in sterile vials as a solution, suspension, gel, emulsion, solid, or as a dehydrated or lyophilized powder. In certain embodiments, such formulations can be stored either in a ready-to-use form or in a form (e.g., lyophilized) that is reconstituted prior to administration.

In certain embodiments, kits are provided for producing a single-dose administration unit (i.e., a dosage unit form). In certain embodiments, the kit can contain both a first container having a dried nucleotide or therapeutic and a second container having an aqueous formulation. In certain embodiments, kits containing single and multi-chambered pre-filled syringes are included.

In certain embodiments, the effective amount of a pharmaceutical composition comprising compositions described herein to be employed therapeutically depends, for example, upon the therapeutic context and objectives. One skilled in the art will appreciate that the appropriate dosage levels for treatment, according to certain embodiments, vary depending, in part, upon the nucleotide or small molecule or other therapeutic delivered, the indication for which therapeutic is being used, the route of administration, and the size (body weight, body surface or organ size) and/or condition (the age and general health) of the patient. The clinician can titer the dosage and modify the route of administration to obtain the optimal therapeutic effect.

The clinician also selects the frequency of dosing, taking into account the pharmacokinetic parameters of the micro-organisms described herein in the formulation used. In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In certain embodiments, the route of administration of the pharmaceutical composition is in accord with known methods, e.g., intravitreally, by subconjunctival injection, by topical (eye drop) administration, orally, through injection by intravenous, intraperitoneal, intracerebral (intra-parenchymal), intracerebral, intraventricular, intramuscular, subcutaneously, intra-ocular, intraarterial, intraportal, or intralesional routes, by sustained release systems or by implantation devices, or other administration routes as described herein. In certain embodiments, the compositions can be administered by bolus injection or continuously by infusion, or by implantation device. In certain embodiments, individual elements of a combination therapy may be administered by different routes.

In certain embodiments, the composition can be administered locally, e.g., during surgery or topically. Optionally local administration is via implantation of a membrane, sponge, or another appropriate material onto which the desired molecule has been absorbed or encapsulated. In certain embodiments, where an implantation device is used, the device can be implanted into any suitable tissue or organ, and delivery of the desired molecule can be via diffusion, timed-release bolus, or continuous administration.

### III. METHODS OF USE AND TREATMENT

As described herein, the present disclosure provides a method of treating a subject with a disorder characterized by, for example, retinal ganglion cell death or retinal cell axonal degeneration, for example (such as those occurring with injuries, diseases and/or disorders as described herein, comprising administering to the subject a therapeutically effective amount of compositions (i.e., those comprising, consisting essentially of, or consisting of therapeutics described herein) according to the present disclosure. In some embodiments, the subject has or is determined to have a disorder causing, for example, retinal ganglion cell death and/or retinal ganglion cell axonal degeneration.

The compositions described herein are useful in, inter alia, methods for treating a disorder in a subject. In some embodiments, the subject has or is suspected to have one or more of: Traumatic Optic Neuropathy (TON), Glaucoma, juvenile glaucoma, Leber's Hereditary Optic Neuropathy, Optic Neuritis, Ischemic Optic Neuropathy, Diabetic Retinopathy, Retinal Occlusion, Anterior Ischemic Optic Neuropathy (AION), Infectious Neuropathies, Neuromyelitis optica (NMO), Behçet's disease, optic nerve hypoplasia, Central or branch retinal artery occlusion (CRAO, BRAO), Radiation Optic Neuropathy, Optic nerve glioma, Age-related macular degeneration (AMD). In some embodiments, the subject is diagnosed with a traumatic optic neuropathy. In some embodiments, the subject is a human that is suspected of having traumatic optic neuropathy.

The compositions can be administered to a subject, e.g., a human subject, using a variety of methods that depend, in part, on the route of administration. The route can be, e.g., intravitreal administration or injection, intravenous injection or infusion (IV), subcutaneous injection (SC), intraperitoneal (IP) injection, intramuscular injection (IM), intradermal injection (ID), subcutaneous, transdermal, intracavity, oral, or intrathecal injection (IT). The injection can be in a bolus or a continuous infusion.

Treating or treatment of any disease or disorder refers to ameliorating a disease or disorder that exists in a subject or a symptom thereof, in particular, ameliorating symptoms of disorders as described herein. The term ameliorating refers to any therapeutically beneficial result in the treatment of a disease state, e.g., a reduction in any one or more symptoms of the disorder, in particular, those that are related to retinal ganglion cell death and/or retinal ganglion cell axonal degeneration.

Thus, treating or treatment includes ameliorating at least one parameter or symptom. Treating or treatment includes modulating the disease or disorder, either physically (e.g., stabilization of a discernible symptom) or physiologically (e.g., stabilization of a physical parameter) or both. Thus, in the disclosed methods, treatment can refer to a 10%, 20%, 30%, 40%, 50%, 60%, 70%, 80%, 90%, or 100% reduction in the severity of an established disease or condition or symptom of the disease or condition. For example, a method for treating a disorder as described herein in a subject by administering a composition as described in this disclosure is considered to be a treatment or therapeutic, for example, if there is a 10% improvement according to the measured or observed parameter in a subject as compared to a control. Thus, the reduction can be a 10%, 20%, 30%, 40%, 50%, 60%, 70%, 80%, 90%, 100%, or more (or any percent improvement in between 10% and 100%) as compared to native or control levels. It is understood that treatment does not necessarily refer to a cure or complete ablation of the disease, condition, or symptoms of the disease or condition.

Described herein are therapeutically effective amounts as defined previously. A therapeutically effective amount is not, however, a dosage so large as to cause adverse side effects. A suitable dose capable of ameliorating a disorder as described herein in a subject, can depend on a variety of factors including the particular construct used and whether it is used concomitantly with other therapeutic agents. Generally, a therapeutically effective amount may vary with the subject's age, condition, and sex, as well as the extent of the disease in the subject and can be determined by one of skill in the art. Other factors can include, e.g., other medical disorders concurrently or previously affecting the subject, the general health of the subject, the genetic disposition of the subject, diet, time of administration, rate of excretion, drug combination, and any other additional therapeutics that are administered to the subject. It should also be understood that a specific dosage and treatment regimen for any particular subject also depends upon the judgment of the treating medical practitioner (e.g., doctor or nurse). A therapeutically effective amount is also one in which any toxic or detrimental effects of the composition are outweighed by the therapeutically beneficial effects. The dosage of the therapeutically effective amount may be adjusted by the individual physician or veterinarian in the event of any complication.

A pharmaceutical composition can include a therapeutically effective amount of any one or more therapeutics described herein. Such effective amounts can be readily determined by one of ordinary skill in the art as described above, embodiments are given throughout the present disclosure.

Suitable human doses of any of the therapeutics described herein can further be evaluated in, e.g., Phase I dose escalation studies. See, e.g., van Gurp et al. (2008) Am J Transplantation 8(8):1711-1718; Hanouska et al. (2007) Clin Cancer Res 13(2, part 1):523-531; and Hetherington et al. (2006) Antimicrobial Agents and Chemotherapy 50(10): 3499-3500.

Toxicity and therapeutic efficacy of such therapeutics can be determined by known pharmaceutical procedures in cell cultures or experimental animals (e.g., animal models of any of the cancers described herein). These procedures can be used, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. A composition comprising therapeutics that exhibit a high therapeutic index is preferred. While therapeutics and nucleotides in particular that may exhibit toxic side effects may be used, care should be taken to design a delivery system that targets such constructs to the site of affected tissue and to minimize potential damage to normal cells and, thereby, reduce side effects.

The data obtained from the cell culture assays and animal studies can be used in formulating a range of dosage for use in humans. The dosage of a therapeutic lies generally within a range of circulating concentrations of the therapeutics that include the ED₅₀ with little or no toxicity. The dosage may vary within this range depending upon the dosage form employed and the route of administration utilized. For therapeutics herein, the therapeutically effective dose can be estimated initially from cell culture assays, for example. A dose can be formulated in animal models to achieve a circulating plasma concentration range that includes the EC₅₀ (i.e., the concentration of the construct—e.g., vector—which achieves a half-maximal inhibition of symptoms) as determined in cell culture. Such information can be used to more accurately determine useful doses in humans. Levels in plasma may be measured, for example, by high performance liquid chromatography. In some embodiments, e.g., where local administration is desired, cell culture or animal models can be used to determine a dose required to achieve a therapeutically effective concentration within the local site.

In some embodiments, a composition or component of a composition described herein can be administered to a subject as a monotherapy. Alternatively, the composition or component of a composition described herein can be administered in conjunction with other therapies for the disorder (combination therapy). For example, the composition can be administered to a subject at the same time, prior to, or after, a second therapy. In some embodiments, the composition or component of a composition described herein, and the one or more additional active agents are administered at the same time. Optionally, the composition or component of a composition described herein is administered first in time and the one or more additional active agents are administered second in time. In some embodiments, the one or more additional active agents are administered first in time and the therapeutic composition or component of a composition described herein is administered second in time. Optionally, the composition or component of a composition described herein, and the one or more additional agents are administered simultaneously in the same or different routes.

A composition as described herein can replace or augment a previously or currently administered therapy, such as previously prescribed antibiotic, steroid, neuroprotective agent, or anti-inflammatory.

Monitoring a subject (e.g., a human patient) for an improvement of symptoms of disorder, as defined herein, means evaluating the subject for a change in a given parameter or symptom exhibited by the subject by a clinician in a social setting or self-reporting by the patient. In some embodiments, the evaluation is performed at least one (1) hour, e.g., at least 2, 4, 6, 8, 12, 24, or 48 hours, or at least 1 day, 2 days, 4 days, 10 days, 13 days, 20 days or more, or at least 1 week, 2 weeks, 4 weeks, 10 weeks, 13 weeks, 20 weeks or more, after an administration. The subject can be evaluated in one or more of the following periods: prior to beginning of treatment; during the treatment; or after one or more elements of the treatment have been administered. Evaluation can include evaluating the need for further treatment, e.g., evaluating whether a dosage, frequency of administration, or duration of treatment should be altered. It can also include evaluating the need to add or drop a selected therapeutic modality.

In certain embodiments, the effective amount of a pharmaceutical composition comprising one or more therapeutics of the present disclosure to be employed therapeutically depends, for example, upon the therapeutic context and objectives. One skilled in the art will appreciate that the appropriate dosage levels for treatment, according to certain embodiments, vary depending, in part, upon the molecule delivered, the indication for which a micro-organism is being used, the route of administration, and the size (body weight, body surface or organ size) and/or condition (the age and general health) of the patient. The clinician can titer the dosage and modify the route of administration to obtain the optimal therapeutic effect.

The clinician also selects the frequency of dosing, taking into account the pharmacokinetic parameters of the active components in the formulation used. Such pharmacokinetic parameters are well known in the art, i.e., the rate of absorption, bioavailability, metabolism, clearance, and the like (see, e.g., Hidalgo-Aragones (1996) J. Steroid Biochem. Mol. Biol. 58:611-617; Groning (1996) Pharmazie 51:337-341; Fotherby (1996) Contraception 54:59-69; Johnson (1995) J. Pharm. Sci. 84:1144-1146; Rohatagi (1995) Pharmazie 50:610-613; Brophy (1983) Eur. J. Clin. Pharmacol. 24:103-108; the latest Remington's, supra). In certain embodiments, a clinician administers the composition until a dosage is reached that achieves the desired effect. In certain embodiments, the composition can therefore be administered as a single dose or as two or more doses (which may or may not contain the same amount of the desired molecule) over time, or as a continuous infusion via, for example, an implantation device or catheter. Further refinement of the appropriate dosage is routinely made by those of ordinary skill in the art and is within the ambit of tasks routinely performed by them. In certain embodiments, appropriate dosages can be ascertained through use of appropriate dose-response data.

In some cases, the dosage (of the active component[s] or compositions as described herein) ranges from a therapeutically-effective amount (or prophylactically-effective amount) of about 1 mg/kg to about 10 mg/kg. In certain examples, the compositions thereof can be administered once every other day at least four times. An exemplary treatment regime may include administration once per day, once per week, twice a week, once every two weeks, once every three weeks, once every four weeks, once a month, once every 3 months, or once every three to 6 months. In some cases, the treatment comprises administering a composition according to one of the aforementioned dosing regimens for a first period and another of the aforementioned dosing regimens for a second period. In some cases, the treatment discontinues for a period of time before the same or a different dosing regimen resumes. For example, a patient may be on a dosing regimen for two weeks, off for a week, on for another two weeks, and so on. In embodiments, treatments such as disulfiram can have a dosage range from about 40 to about 80 mg/kg; 2-BP and the like can have a dosage range of about 2 to about 4 g/kg. For AAV virus, about 5×10\hat{ }9 to about 1×10\hat{ }10 gc/eye can be given.

As would be understood by the skilled artisan, in vivo assessment of improved cell survival can be conducted at least by the following, for example and without intending to be limiting: quantification of RGC survival by staining with RGC specific markers (for example, RBPMS, BRN3A); TUNEL staining to directly detect dying RGCs; and measuring the axon degeneration by staining with axon markers, TUJ1. Or GFP labeling.

As would be understood by the skilled artisan, to assess axonal degeneration, methodologies that can be used include, for example and without intending to be limiting: IF staining with axon markers (for example, TUJ1 and Neurofilament (NF)); para-phenylenediamine (PPD) staining; and intravitreal injection of fluorescent dyes or proteins, for example, Cholera Toxin Subunit B CF® Dye Conjugates, or GFP

### IV. KITS AND PACKAGING

Described herein are kits comprising nucleotides, small molecules, and other therapeutics contemplated by the present disclosure, in addition to containers for storage and/or use, and instructions for use. The kit can be a package which houses a container which contains compounds of the disclosure or formulations of the disclosure and also houses instructions for administering the compounds or formulations to a subject. The disclosure further relates to a commercial package comprising compounds of the disclosure or formulations of the disclosure together with instructions for simultaneous, separate or sequential use. In particular a label may include amount, frequency, and method of administration.

The disclosure also provides a pharmaceutical pack or kit comprising one or more containers filled with one or more of the ingredients of a composition of the disclosure to provide a therapeutic effect. Associated with such container(s) can be various written materials such as instructions for use, or a notice in the form prescribed by a governmental agency regulating the labeling, manufacture, use or sale of pharmaceuticals or biological products, which notice reflects approval by the agency of manufacture, use, or sale for human administration.

The disclosure also relates to articles of manufacture and kits containing materials useful for treating a disease disclosed herein. An article of manufacture may comprise a container with a label. Examples of suitable containers include bottles, vials, and test tubes, or a delivery device such as a nebulizer, which may be formed from a variety of materials including glass and plastic. A container holds compounds of the disclosure or formulations of the disclosure which are effective for treating a disease disclosed herein. The label on the container indicates that the compounds of the disclosure or formulations of the disclosure are used for treating a disease disclosed herein and may also indicate directions for use. In aspects of the disclosure, a medicament or formulation in a container may comprise any of the medicaments or formulations disclosed herein.

The disclosure also contemplates kits comprising one or more of compounds, nucleotides, or other therapeutics of the disclosure. In aspects of the disclosure, a kit of the disclosure comprises a container described herein. In particular aspects, a kit of the disclosure comprises a container described herein and a second container comprising a buffer. A kit may additionally include other materials desirable from a commercial and user standpoint, including, without limitation, buffers, diluents, filters, needles, syringes, and package inserts with instructions for performing any methods disclosed herein (e.g., methods for treating a disease disclosed herein). A medicament or formulation in a kit of the disclosure may comprise any of the formulations or compositions disclosed herein.

The compositions (i.e., those comprising, consisting essentially of, or consisting of nucleotides, small molecules, and other therapeutics described herein) can be utilized in the preparation of a kit. In some embodiments, kits are provided for carrying out any of the methods described herein. The kits of this disclosure may comprise a carrier container being compartmentalized to receive in close confinement one or more containers such as vials, tubes, and the like, each of the containers comprising one of the separate elements to be used in the methods.

In some instances, one of the containers may comprise a composition as described in this disclosure that is, or can be, detectably labeled. The kit may also have containers containing buffer(s) and/or a container comprising a reporter-means, such as a biotin-binding protein, such as avidin or streptavidin, bound to a reporter molecule, such as an enzymatic or fluorescent label. In some embodiments, the kit comprises separate containers containing compositions described herein and a detectable label.

A composition as described in this disclosure for use in targeting Gasdermin E in subjects may be delivered in a pharmaceutical package or kit to doctors and patients having or suspected of having a disorder as described herein. Such packaging is intended to improve patient convenience and compliance with the treatment plan. Typically, the packaging comprises paper (cardboard) or plastic. In some embodiments, the kit or pharmaceutical package further comprises instructions for use (e.g., for administering according to a method as described herein).

In some embodiments, a pharmaceutical package or kit comprises unit dose forms of a composition or components of compositions described herein. In some embodiments, the pharmaceutical package or kit further comprises unit dose forms of one or more of an additional therapeutic, for example, another medicament used for treatment of a disorder in a patient.

In one embodiment, the kit or pharmaceutical package comprises a composition as described herein in a defined, therapeutically effective dose in a single unit dosage form or as separate unit doses. The dose and form of the unit dose (e.g., tablet, capsule, immediate release, delayed release, etc.) can be any doses or forms as described herein.

In one embodiment, the kit or pharmaceutical package includes doses suitable for multiple days of administration, such as one week, one month, or three months.

In certain embodiments, kits are provided for producing a single-dose administration unit. In certain embodiments, kits containing single or multi-chambered pre-filled syringes are included. In certain embodiments, kits containing one or more containers of a formulation described in this disclosure are included.

### V. EXAMPLES

Now having described the embodiments of the disclosure, in general, the examples describe some additional embodiments. While embodiments of the present disclosure are described in connection with the example and the corresponding text and figures, there is no intent to limit embodiments of the disclosure to these descriptions. On the contrary, the intent is to cover all alternatives, modifications, and equivalents included within the spirit and scope of embodiments of the present disclosure.

The following examples are put forth so as to provide those of ordinary skill in the art with a complete disclosure and description of how to perform the methods and use the compositions and compounds disclosed and claimed herein. Efforts have been made to ensure accuracy with respect to numbers (e.g., amounts, temperature, etc.), but some errors and deviations should be accounted for. Unless indicated otherwise, parts are parts by weight, temperature is in ° C., and pressure is in atmosphere. Standard temperature and pressure are defined as 25° C. and 1 atmosphere.

### Example 1: Gasdermin E Expression Correlates with RGC Vulnerability

Gasdermin proteins formed oligomeric pores and are associated with pyroptosis, a process linked to various neurodegenerative conditions⁴.

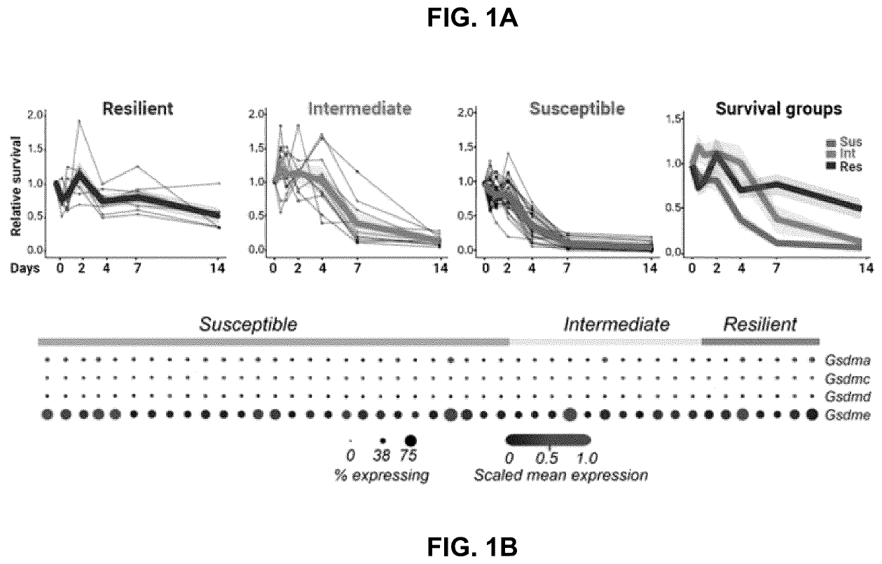

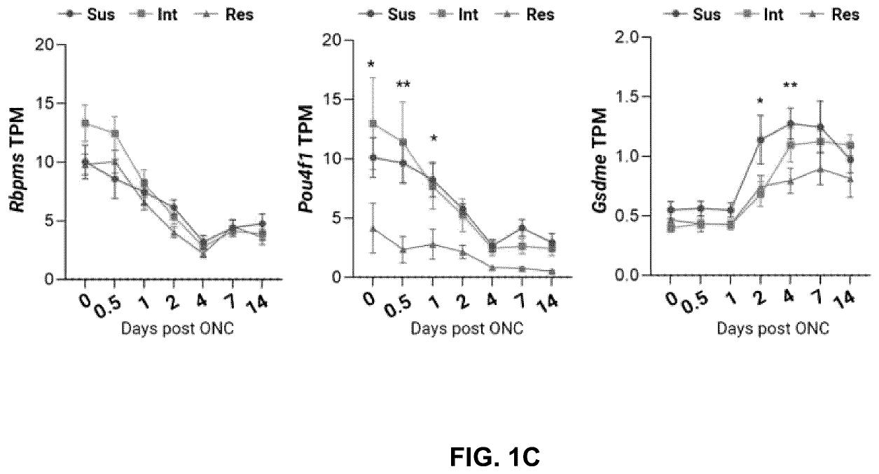

The scRNA-seq data⁵ from injured RGC indicate that Gsdme is the most abundantly expressed gasdermin gene across the RGC types, and its mRNA levels significantly enriched in vulnerable RGC populations following optic nerve injury (FIGS. 1A-1C).

The biological mechanisms, states, and specific impacts of gasdermin E activation on RGC death remain entirely unknown. The present example examples the effect of knockdown, ablation, activity reduction, and/or inhibition of Gasdermin E in RGCs.

First, it was found gasdermin E expression correlates with RGC vulnerability (FIGS. 1A-1C). FIG. 1A shows a classification of three survival groups (Resilient, Intermediate and Susceptible) of mouse RGC based on the pattern of cell loss across time after optic nerve crush. FIG. 1B is a dot plot of the single cell expression levels of murine gasdermin genes (a, c, d, e) in three survival groups of RGC. FIG. 1C shows single cell expression levels of RGC signature genes (Rbpms, Pou4f1) and Gsdme in RGCs across time after ONC. Two-way ANOVA with tukey's multiple comparisons test. *P<0.05. **P<0.01.

### Example 2: Methods/Experimental Design of an Animal Model of Traumatic Optic Neuropathy (TON)

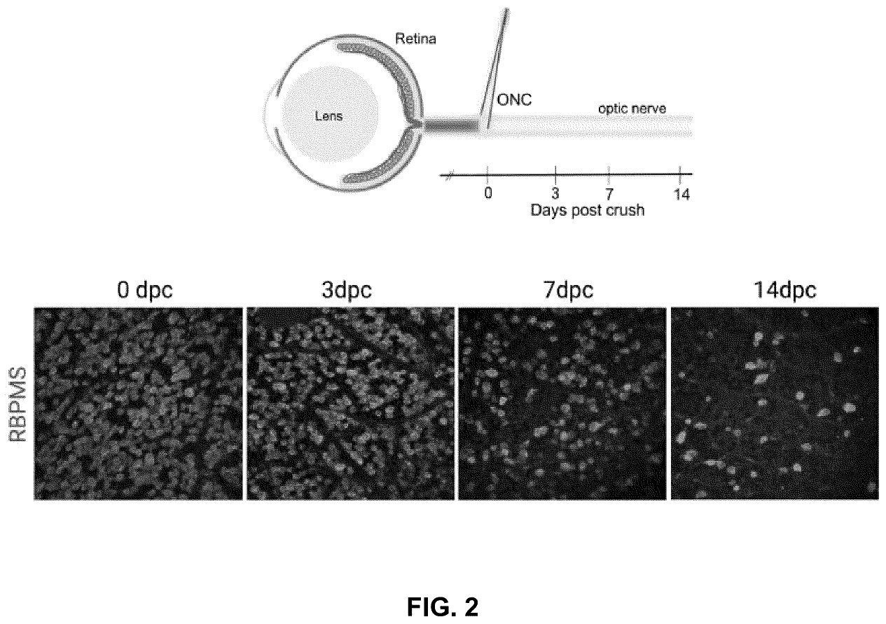

In the mouse model of TON, the optic nerve of the right eye is crushed approximately 2 mm behind the eyeball using #5 cross-action self-closing forceps (FIG. 2).

RGC survival was assessed using immunofluorescent staining of the RGC-specific markers, RBPMS or Brn3a.

Single-cell RNA sequencing (scRNA-seq) data⁵ were used to assess the expression levels of gasdermin genes implicated in RGC death.

Immunofluorescence (IF) staining and immunoblotting were performed to evaluate the levels of gasdermin protein and its proteolytic processing.

NIrp3^{-/-}, NIrc4^{-/-}, Casp1^{-/-}Casp4^{-/-} and Gsdme^{-/-} mice were used to study the role of the inflammasome pathway and gasdermin E in RGC degeneration.

Intravitreous injection of AAV-GFP-shRNA virus to test the therapeutic efficacy of AAV targeting gasdermin E in treating RGC degeneration of TON.

Aspects of the mouse model of TON are further illustrated in FIG. 2. The RGC specific marker, RBPMS, was used to label RGCs in flat-mounted retinas at different time points after optic nerve crush. Scale bar, 10 μm.

### Example 3: Neuronal Gasdermin E Activation in Experimental TON

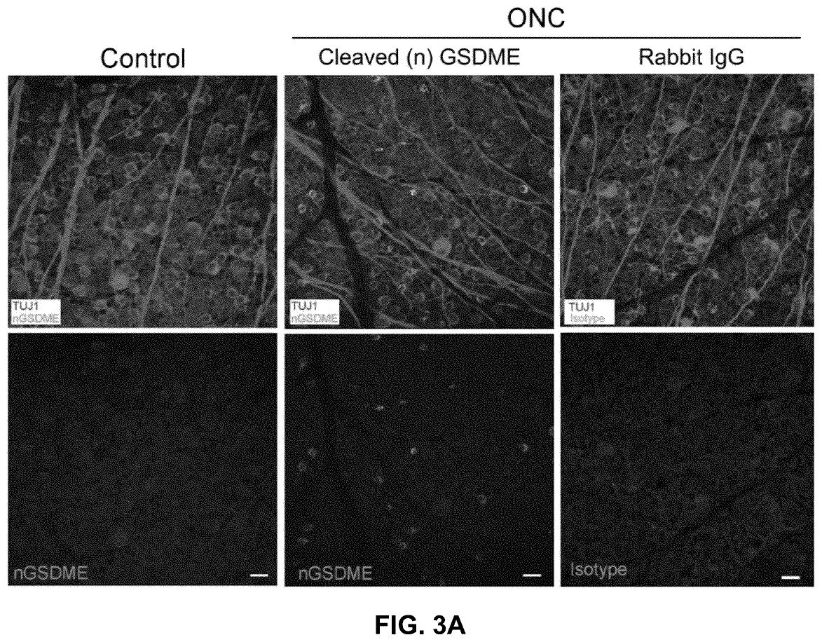

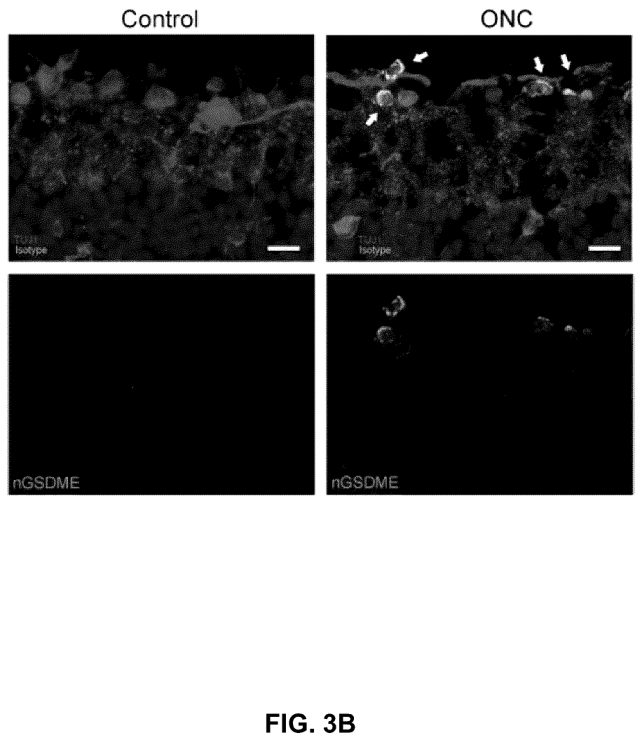

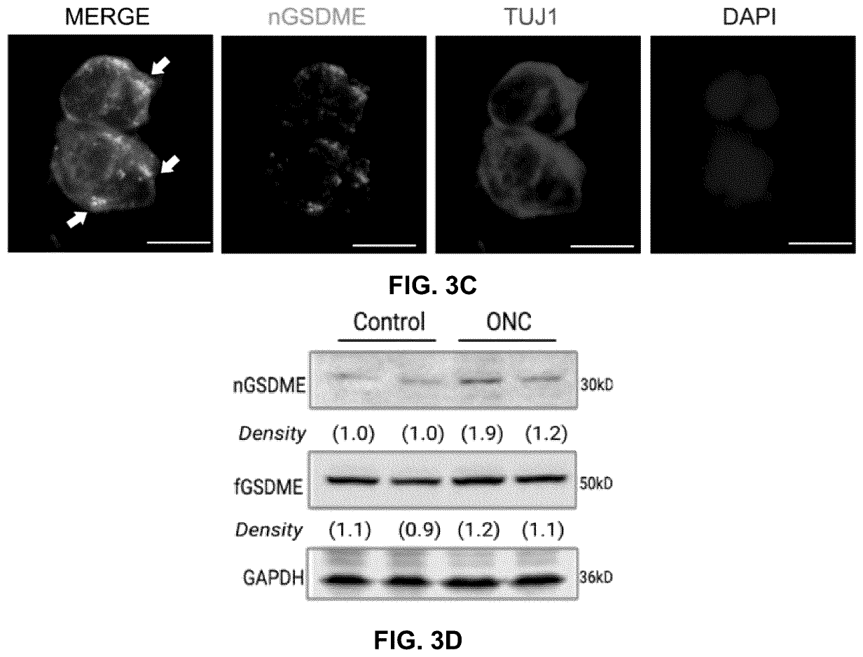

FIGS. 3A-3D illustrate aspects of neuronal gasdermin E activation in RGC degeneration. FIG. 3A illustrates IF staining of RGC specific marker, TUJ1, cleaved gasdermin E and Rabbit IgG isotype control with retinal whole-mounts from wild-type mice at 3 days post-ONC. FIG. 3B illustrates IF staining of RGC specific marker, TUJ1, cleaved gasdermin E with cryosections of wild-type mice at 3 days post-ONC. FIG. 3C shows confocal images of gasdermin E activation and pore formation in TUJ1+ RGCs at 3 days post-ONC. FIG. 3D illustrates immunoblotting and densitometry analysis of nGSDME, fGSDME and GAPDH with retina lysates collected at 3 days post-ONC.

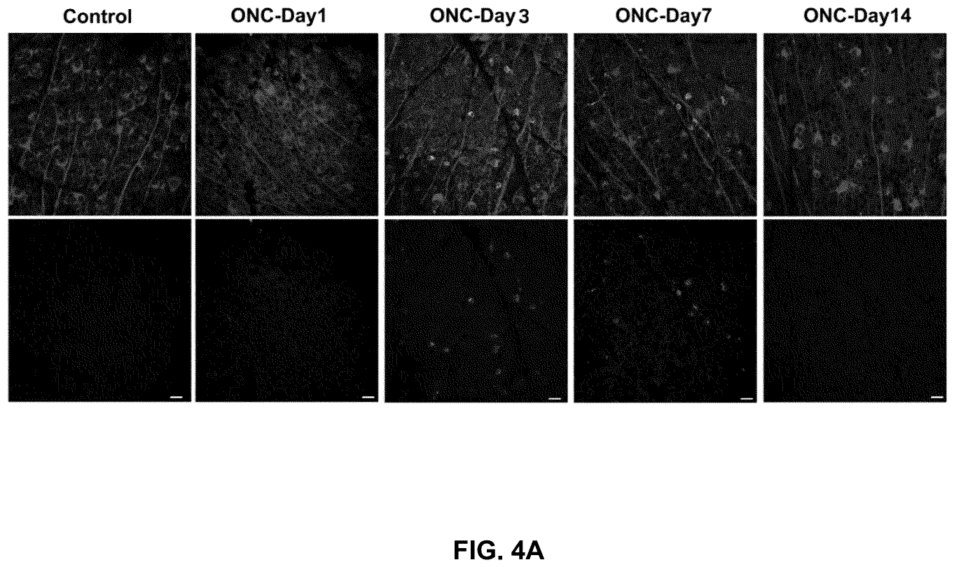

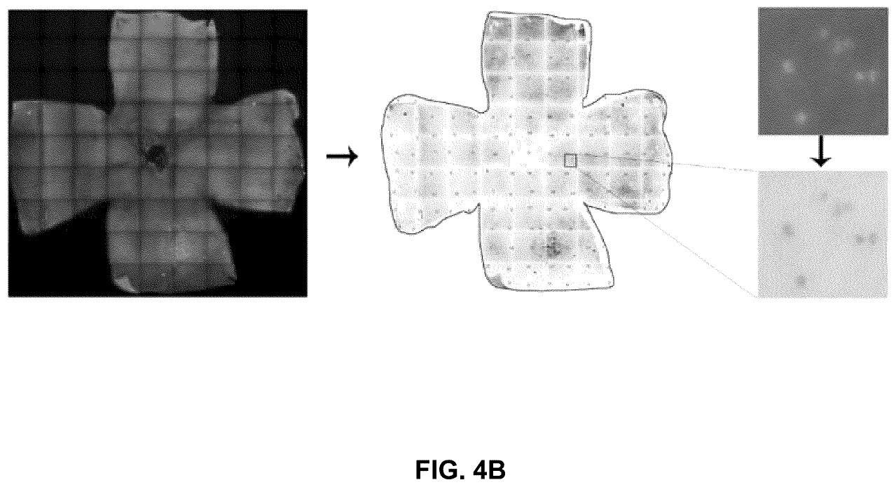

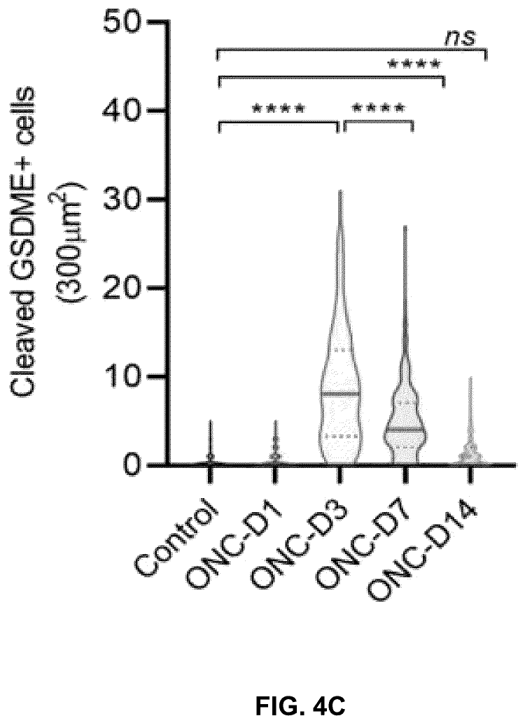

FIGS. 4A-4C illustrate gasdermin E activation occurring at the early stage of RGC degeneration. FIG. 4A is immunofluorescence staining of TuJ1 (class III beta-tubulin) and cleaved gasdermin E (nGSDME) with retina whole-mounts from wild-type mice at various time points post-ONC. FIG. 4B is an illustration of the methods employed using Image J to quantify the nGSDME positive RGC with retinal whole-mounts. FIG. 4C depicts quantification of nGSDME+RGC with retina collected at various time points post-ONC. N=3, One-way ANOVA with Tukey's multiple comparisons test. ****P<0.0001. Scale bar, 10 μm.

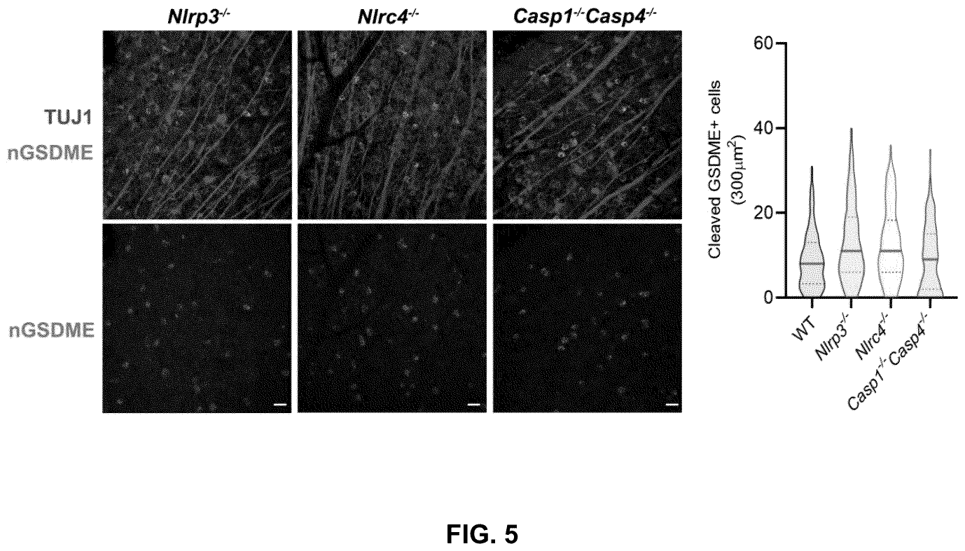

FIG. 5 shows that an inflammasome pathway is dispensable for neuronal gasdermin E activation in RGC. Immunofluorescence staining (left panels) and quantification (rightmost panel) of TuJ1 (class III beta-tubulin) and cleaved gasdermin E (nGSDME) with retinal whole-mounts from NIrp3^{-/-}, NIrc4^{-/-} and Casp1^{-/-}Casp4^{-/-} mice after optic nerve crush. N=3, One-way ANOVA with tukey's multiple comparisons test. Scale bar, 10 μm.

### Example 4: Gasdermin E Blockage Protects RGCs in Experimental TON

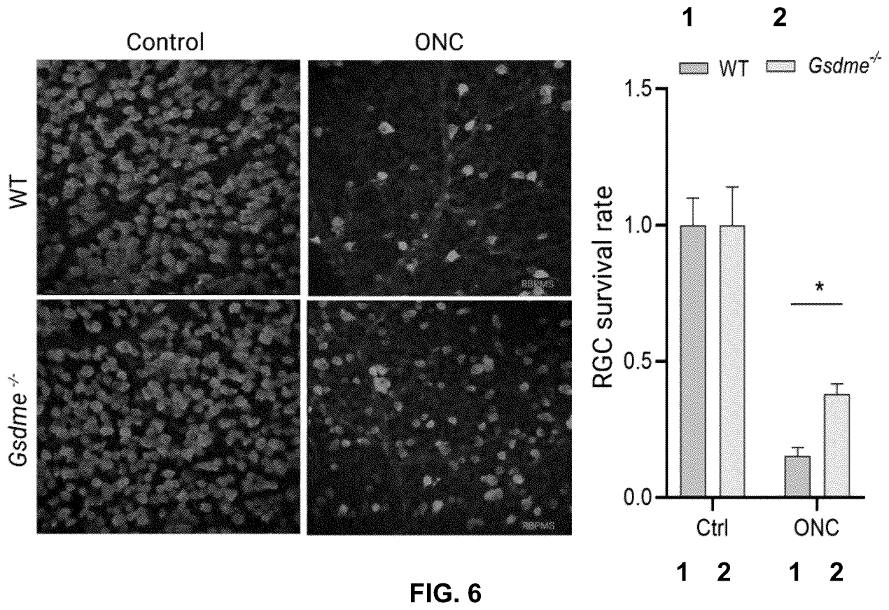

FIG. 6 shows that genetic ablation of GSDME prolongs RGC survival after optic nerve injury. Immunolabeling (left panels) and quantification (graph in rightmost panel) of RGC with retinal whole-mounts at 14 days post-ONC. N=3, Unpaired t test. *P<0.05. Scale bar, 10 μm.

### Example 5: Gene Therapy Targeting Gasdermin E Alleviates RGC Degeneration in Experimental TON

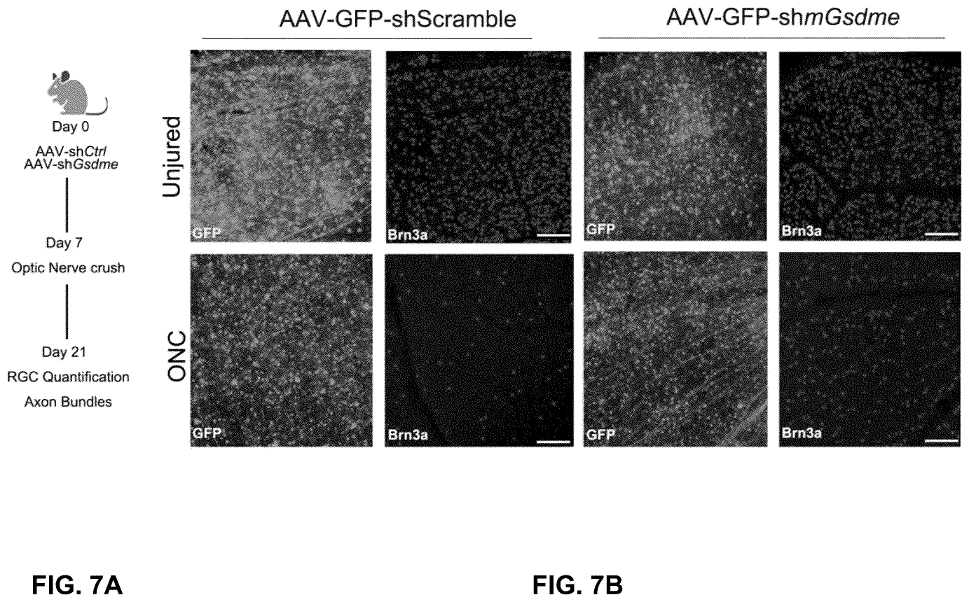

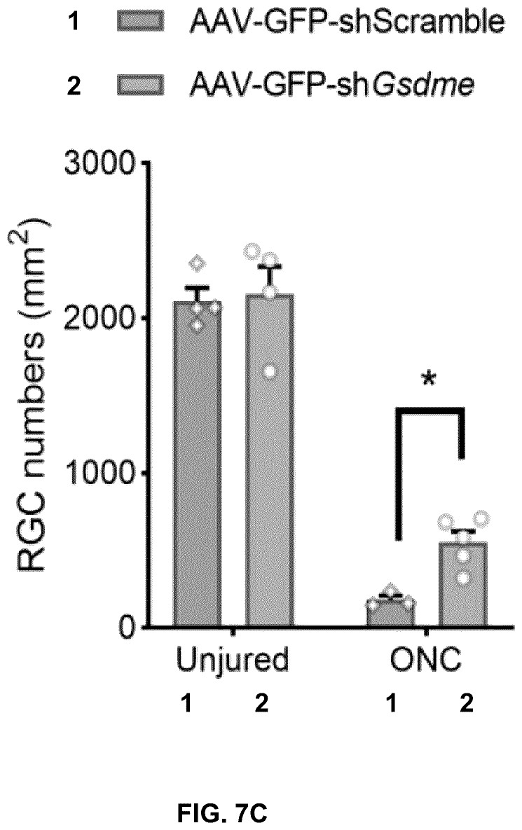

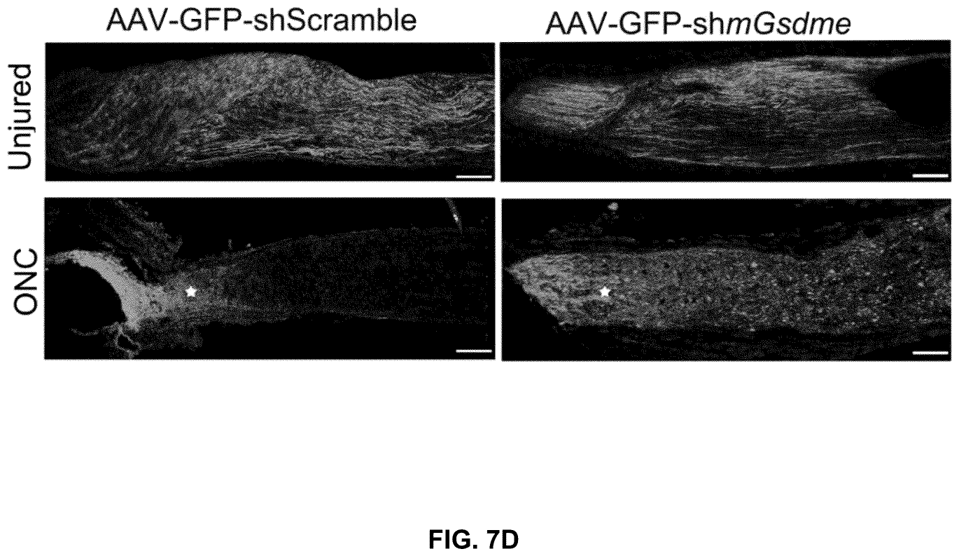

FIGS. 7A-7D show that an AAV targeting gasdermin E prolongs RGC survival and halts axon degeneration after optic nerve injury. FIG. 7A shows an embodiment of an experimental design to assess in vivo therapeutic efficacy of AAV-mediated Gasdermin E knockdown in treating TON. FIG. 7B illustrates immunofluorescence (IF) staining of RGC marker Brn3a, and (FIG. 7C) quantification of RGCs in retinas treated with AAV-GPP-shCtrl or AAV-GPP-shGsdme 14 days post-ONC (N=3; unpaired t test, P<0.05). Scale bar, 100 μm. (FIG. 7D) Confocal images of anterograde GFP tracing of RGC axons in the optic nerve 14 days post-ONC. Scale bar, 100 μm.

### Example 6: Additional Discussion Relating to Examples 1-5

### Introduction:

Traumatic optic neuropathy (TON) is the leading neuro-ophthalmic injury in U.S. soldiers, often causing vision loss or permanent blindness, significantly impacting veterans' quality of life^{1,2} Current treatments, including high-dose corticosteroids and surgical decompression are largely ineffective due to limited understanding of the fundamental mechanism underlying retinal ganglion cell (RGC) death³.

### Methods:

Single-cell RNA sequencing (scRNA) sequencing database, immunofluorescence (IF) staining, and immunoblotting were used to assess gasdermin E mRNA expression and proteolytic activation. NIrp3^{-/-}, NIrc4^{-/-}, Casp1^{-/-}Casp4^{-/-} and Gsdme^{-/-} mice were employed to study inflammasome pathways in RGC degeneration. AAV-shRNA was used to evaluate the in vivo therapeutic efficacy of targeting gasdermin E in treating RGC degeneration of TON.

### Results:

Robust neuronal activation of gasdermin E occurs independently of its inflammasome signaling pathway after optic nerve injury. Targeting gasdermin E via AAV significantly extends RGC survival and prevents axon degeneration in a mouse model of TON

As is demonstrated and can be deduced at least from Examples 1-5 above, Optic nerve injury triggers robust Gasdermin E activation in RGCs.

Neuronal Gasdermin E activation occurs independent of its inflammasome signaling pathway.

Targeting Gasdermin E prolongs RGC survival and halts axon degeneration in the experimental TON.

### Example 7: Embodiment of shRNA Delivery Vector

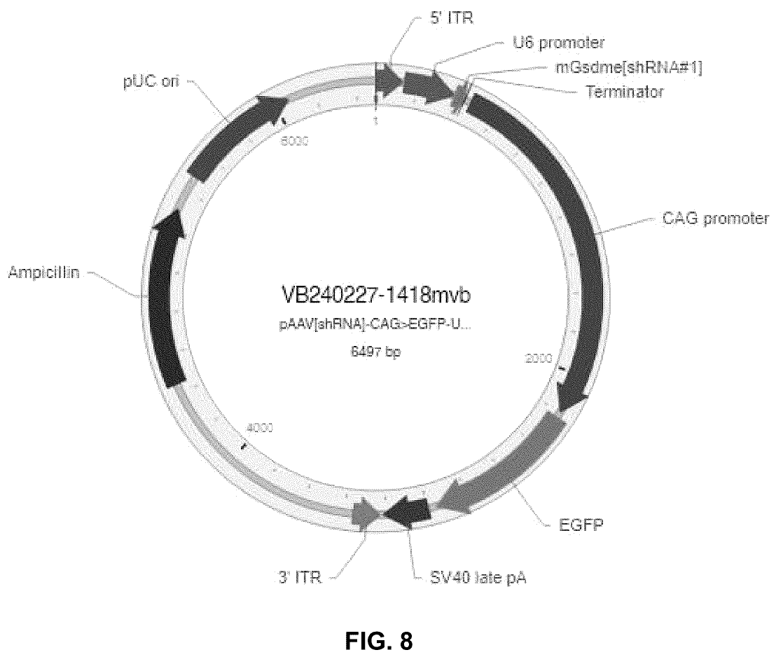

FIG. 8 shows an embodiment of a plasmid vector that can be utilized according to aspects of the present disclosure that can be utilized to deliver a Gasdermin E-targeting nucleotide to a cell, tissue, or subject.

### Example 8: Additional Aspects of the Present Disclosure

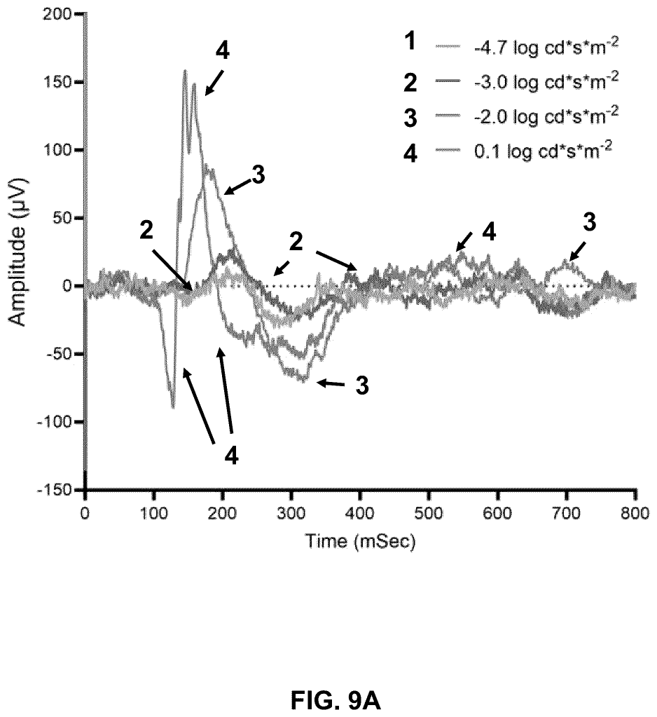

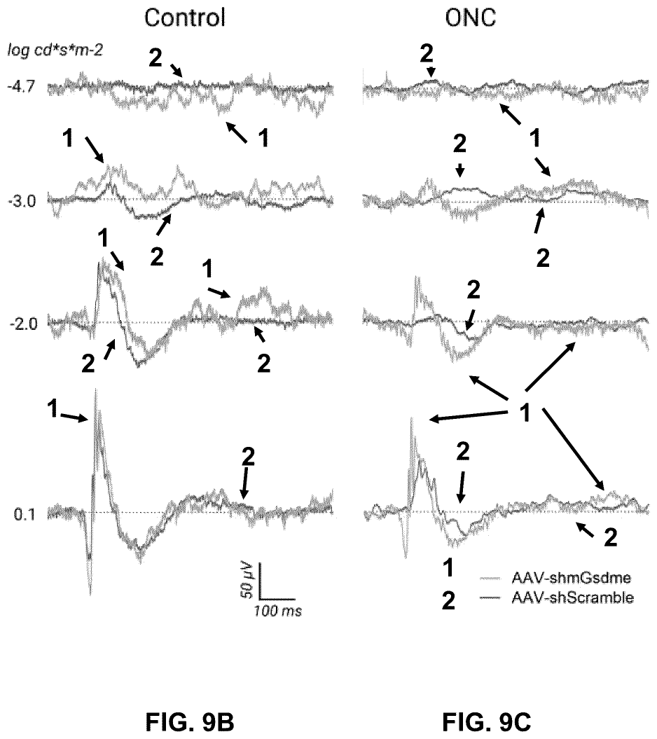

FIGS. 9A-9C. Knockdown of Gsdme preserves visual function. Representative electroretinogram (ERG) waveforms (FIG. 9A) and scotopic threshold response (STR) recordings from control (FIG. 9B) and Gsdme-knockdown mice (FIG. 9C) under various light intensities. Knockdown of Gsdme markedly preserved STR amplitudes compared to controls.

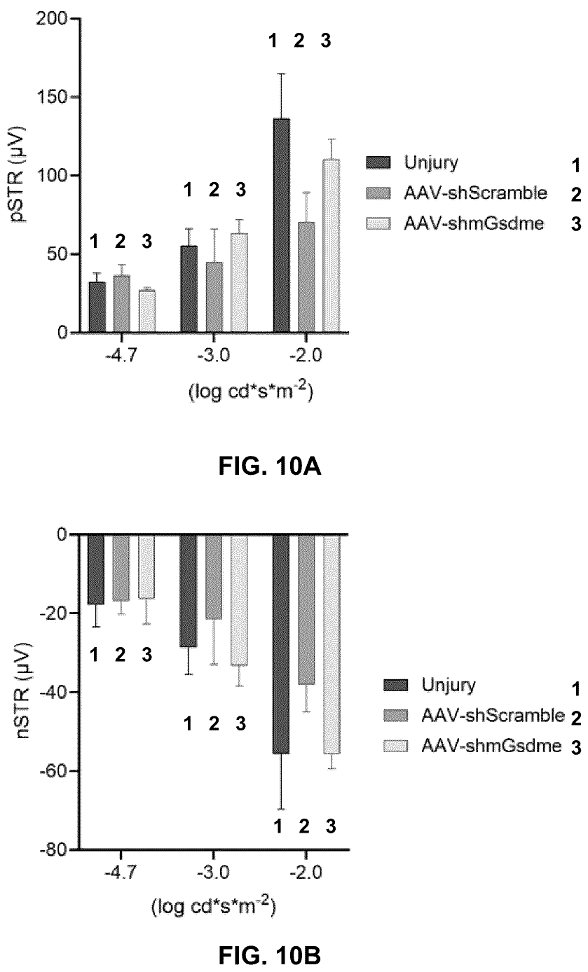

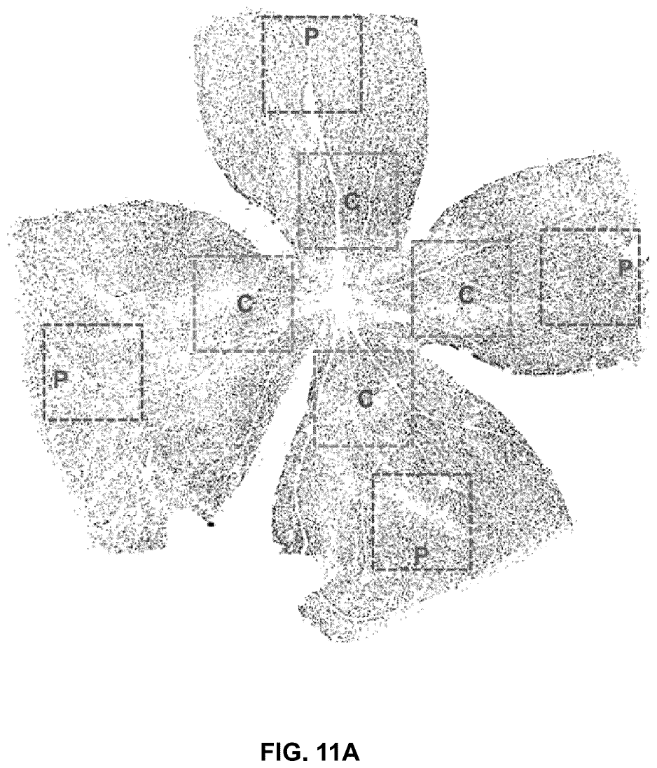

FIGS. 10A-10B. Quantification of scotopic ERG parameters after Gsdme knockdown. Averaged amplitudes of positive STR (pSTR; FIG. 10A) and negative STR (nSTR; FIG. 10B) were analyzed across multiple light intensities. Gsdme knockdown significantly enhanced both pSTR and nSTR compared to scrambled controls.

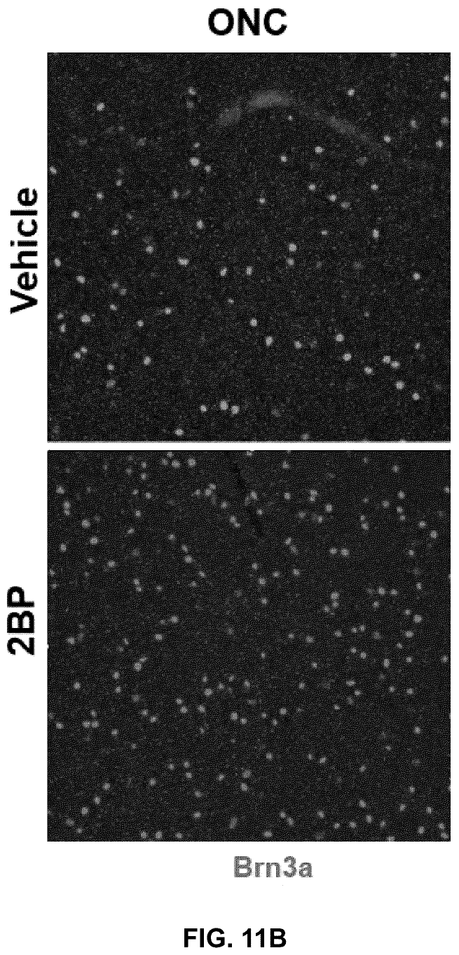

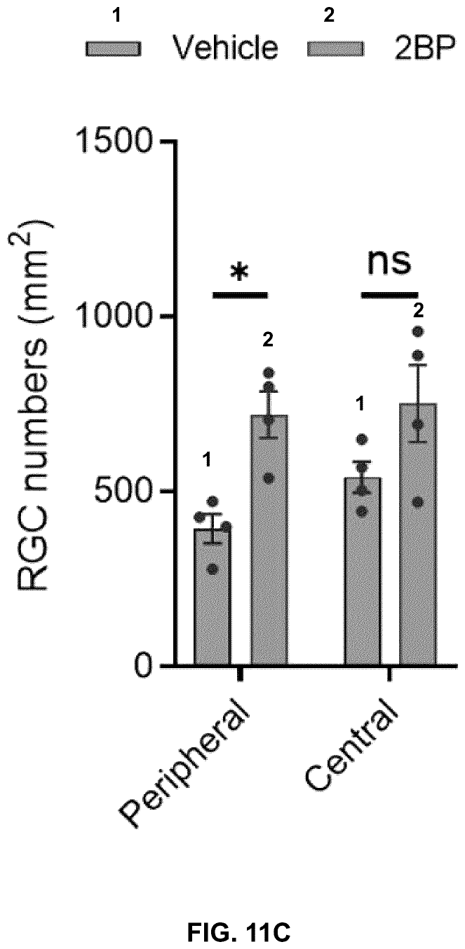

FIGS. 11A-11C. Pharmacological inhibition of GSDME activation by 2-bromopalmitate (2-BP) attenuates optic nerve crush (ONC)-induced retinal ganglion cell (RGC) loss. (FIG. 11A) Experimental design illustrating flat-mounted retina segmentation and RGC counting. (FIG. 11B) Representative retinal flat-mounts immunostained for Brn3a showing RGC survival 14 days after ONC. (FIG. 11C) Quantification of Brn3a⁺ RGC density revealed significant protection in 2-BP-treated eyes compared with vehicle controls.

### REFERENCES RELATED TO THE PRESENT DISCLOSURE

1. Winkler, S. L. et al. Veterans with Traumatic Brain Injury-related Ocular Injury and Vision Dysfunction: Recommendations for Rehabilitation. Optom Vis Sci 99, 9-17 (2022).
2. Harvey, M. M. et al. Ocular Trauma in Operation Iraqi Freedom and Operation Enduring Freedom from 2001 to 2011: A Bayesian Network Analysis. Ophthalmic Epidemiol 28, 312-321, (2021).
3. Blanch, R. J., Joseph, I. J. & Cockerham, K. Traumatic optic neuropathy management: a systematic review. Eye (Lond), (2024).
4. Tran, N. M. et al. Single-Cell Profiles of Retinal Ganglion Cells Differing in Resilience to Injury Reveal Neuroprotective Genes. Neuron 104, 1039-1055, (2019).

INFORMAL SEQUENCE LISTING:

| SEQ | | |
|---|---|---|
| ID NO: | SEQUENCE | DESCRIPTION |
| 1 | GATGATGGAGTATCTGATCTT | Murine GSDME |
| | | shRNA targeting |
| | | sequence 1 |
| 2 | GGGATCCAGACCAAGACTATA | Murine GSDME |
| | | shRNA targeting |
| | | sequence 2 |
| 3 | GTCACCACGGACACCAATGTA | Murine GSDME |
| | | shRNA targeting |
| | | sequence 3 |
| 4 | GGTCAGCGCACTAGCAGAAAT | Murine GSDME |
| | | shRNA targeting |
| | | sequence 4 |
| 5 | GAATGACTCTGATAAGTTACA | optimized shRNA |
| | | sequence for |
| | | targeting human |
| | | GSDME no. 1 |
| 6 | GTGAAATACGAGGGCAAGTTT | optimized shRNA |
| | | sequence for |
| | | targeting human |
| | | GSDME no. 2 |
| 7 | GACCCAGCATCTGAGTTAGGT | optimized shRNA |
| | | sequence for |
| | | targeting human |
| | | GSDME no. 3 |
| 8 | GATGATGGAGTATCTGATCTT | optimized shRNA |
| | | sequence for |
| | | targeting human |
| | | GSDME no. 4 |
| 9 | GCGGTCCTATTTGATGATGAA | optimized shRNA |
| | | sequence for |
| | | targeting human |
| | | GSDME no. 5 |
| 10 | GCATGATGAATGACCTGACTT | optimized shRNA |
| | | sequence for |
| | | targeting human |
| | | GSDME no. 6 |
| 11 | GCTTCTAAGTCTGGTGACAAA | optimized shRNA |
| | | sequence for |
| | | targeting human |
| | | GSDME no. 7 |
| 12 | GCATTCATAGACATGCCAGAT | optimized shRNA |
| | | sequence for |
| | | targeting human |
| | | GSDME no. 8 |
| 13 | CTCTGCCGAGAGAACAATAAA | optimized shRNA |
| | | sequence for |
| | | targeting human |
| | | GSDME no. 9 |
| 14 | TCTGCCGAGAGAACAATAAAT | optimized shRNA |
| | | sequence for |
| | | targeting human |
| | | GSDME no. 10 |
| 15 | AGCCACCAGGTACTCTTTAAA | optimized shRNA |
| | | sequence for |
| | | targeting human |
| | | GSDME no. 11 |
| 16 | GAAAGAGTCTGACCCTTTAAT | optimized shRNA |
| | | sequence for |
| | | targeting human |
| | | GSDME no. 12 |
| 17 | TGGAAGTGATGTATGTTATTT | optimized shRNA |
| | | sequence for |
| | | targeting human |
| | | GSDME no. 13 |
| 18 | TAAATGCTCCTAGAGTATATT | optimized shRNA |
| | | sequence for |
| | | targeting human |
| | | GSDME no. 14 |
| 19 | GTATTCTGGGTATTCTATTAA | optimized shRNA |
| | | sequence for |
| | | targeting human |
| | | GSDME no. 15 |
| 20 | TCTGTCTGATGATGGAGTA | Anti-Gasdermin E |
| | | siRNA sequence |
| | | 1 |
| 21 | TGACATTAGTCTGGAGAGA | Anti-Gasdermin E |
| | | siRNA sequence |
| | | 2 |
| 22 | GAATGACTCTGATAAGTTA | Anti-Gasdermin E |
| | | siRNA sequence |
| | | 3 |
| 23 | CCTACGGTGTCATTGAGTT | Anti-Gasdermin E |
| | | siRNA sequence |
| | | 4 |
| 24 | GCTTCTAAGTCTGGTGACA | Anti-Gasdermin E |
| | | siRNA sequence |
| | | 5 |
| 25 | CCTTGGCGATGTACTCATA | Anti-Gasdermin E |
| | | siRNA sequence |
| | | 6 |
| 26 | GCAGAAGTGTGTGATCTCT | Anti-Gasdermin E |
| | | siRNA sequence |
| | | 7 |
| 27 | TGTGTGATCTCTGAGCACA | Anti-Gasdermin E |
| | | siRNA sequence |
| | | 8 |
| 28 | GCTTCGTGCTCTGTCTGAT | Anti-Gasdermin E |
| | | siRNA sequence |
| | | 9 |
| 29 | TTAGAGAAGTTGATGCTGA | Anti-Gasdermin E |
| | | siRNA sequence |
| | | 10 |
| 30 | CTGCGCGCTCGCTCGCTCACTGAGGCCGCCCGGGCAA | AAV-GFP-shRNA |
| | AGCCCGGGCGTCGGGCGACCTTTGGTCGCCCGGCCTC | plasmid |
| | AGTGAGCGAGCGAGCGCGCAGAGAGGGAGTGGCCAA | |
| | CTCCATCACTAGGGGTTCCTATCGATGAGGGCCTATTT | |
| | CCCATGATTCCTTCATATTTGCATATACGATACAAGGCT | |
| | GTTAGAGAGATAATTGGAATTAATTTGACTGTAAACACA | |
| | AAGATATTAGTACAAAATACGTGACGTAGAAAGTAATAA | |
| | TTTCTTGGGTAGTTTGCAGTTTTAAAATTATGTTTTAAAA | |
| | TGGACTATCATATGCTTACCGTAACTTGAAAGTATTTCG | |
| | ATTTCTTGGCTTTATATATCTTGTGGAAAGGACGAAACA | |
| | CCGGCCGTCAAGAGAACAGTTAATACTCGAGTATTAAC | |
| | TGTTCTCTTGACGGTTTTTGAATTCCAACTTTGTATAGAA | |
| | AAGTTGCTCGACATTGATTATTGACTAGTTATTAATAGTA | |
| | ATCAATTACGGGGTCATTAGTTCATAGCCCATATATGGA | |
| | GTTCCGCGTTACATAACTTACGGTAAATGGCCCGCCTG | |
| | GCTGACCGCCCAACGACCCCCGCCCATTGACGTCAATA | |
| | ATGACGTATGTTCCCATAGTAACGCCAATAGGGACTTTC | |
| | CATTGACGTCAATGGGTGGAGTATTTACGGTAAACTGC | |
| | CCACTTGGCAGTACATCAAGTGTATCATATGCCAAGTAC | |
| | GCCCCCTATTGACGTCAATGACGGTAAATGGCCCGCCT | |
| | GGCATTATGCCCAGTACATGACCTTATGGGACTTTCCTA | |
| | CTTGGCAGTACATCTACGTATTAGTCATCGCTATTACCA | |
| | TGGTCGAGGTGAGCCCCACGTTCTGCTTCACTCTCCCC | |
| | ATCTCCCCCCCCTCCCCACCCCCAATTTTGTATTTATTT | |
| | ATTTTTTAATTATTTTGTGCAGCGATGGGGGCGGGGGGG | |
| | GGGGGGGGGGGGCGCGCCAGGCGGGGCGGGGCGGG | |
| | GCGAGGGGCGGGGCGGGGCGAGGCGGAGAGGTGCG | |
| | GCGGCAGCCAATCAGAGCGGCGCGCTCCGAAAGTTTC | |
| | CTTTTATGGCGAGGCGGCGGCGGCGGCGGCCCTATAA | |
| | AAAGCGAAGCGCGCGGCGGGCGGGAGTCGCTGCGCG | |
| | CTGCCTTCGCCCCGTGCCCCGCTCCGCCGCCGCCTCG | |
| | CGCCGCCCGCCCCGGCTCTGACTGACCGCGTTACTCC | |
| | CACAGGTGAGCGGGCGGGACGGCCCTTCTCCTCCGGG | |
| | CTGTAATTAGCGCTTGGTTTAATGACGGCTTGTTTCTTT | |
| | TCTGTGGCTGCGTGAAAGCCTTGAGGGGCTCCGGGAG | |
| | GGCCCTTTGTGCGGGGGGAGCGGCTCGGGGGGTGCG | |
| | TGCGTGTGTGTGTGCGTGGGGAGCGCCGCGTGCGGCT | |
| | CCGCGCTGCCCGGCGGCTGTGAGCGCTGCGGGCGCG | |
| | GCGCGGGGCTTTGTGCGCTCCGCAGTGTGCGCGAGGG | |
| | GAGCGCGGCCGGGGGCGGTGCCCCGCGGTGCGGGG | |
| | GGGGCTGCGAGGGGAACAAAGGCTGCGTGCGGGGTG | |
| | TGTGCGTGGGGGGGTGAGCAGGGGGTGTGGGCGCGT | |
| | CGGTCGGGCTGCAACCCCCCCTGCACCCCCCTCCCCG | |
| | AGTTGCTGAGCACGGCCCGGCTTCGGGTGCGGGGCTC | |
| | CGTACGGGGCGTGGCGCGGGGCTCGCCGTGCCGGGC | |
| | GGGGGGTGGCGGCAGGTGGGGGTGCCGGGCGGGGC | |
| | GGGGCCGCCTCGGGCCGGGGAGGGCTCGGGGGAGG | |
| | GGCGCGGCGGCCCCCGGAGCGCCGGCGGCTGTCGAG | |
| | GCGCGGCGAGCCGCAGCCATTGCCTTTTATGGTAATCG | |
| | TGCGAGAGGGCGCAGGGACTTCCTTTGTCCCAAATCTG | |
| | TGCGGAGCCGAAATCTGGGAGGCGCCGCCGCACCCCC | |
| | TCTAGCGGGCGCGGGGCGAAGCGGTGCGGCGCCGGC | |
| | AGGAAGGAAATGGGCGGGGAGGGCCTTCGTGCGTCGC | |
| | CGCGCCGCCGTCCCCTTCTCCCTCTCCAGCCTCGGGG | |
| | CTGTCCGCGGGGGGACGGCTGCCTTCGGGGGGGACG | |
| | GGGCAGGGCGGGGTTCGGCTTCTGGCGTGTGACCGG | |
| | CGGCTCTAGAGCCTCTGCTAACCATGTTCATGCCTTCTT | |
| | CTTTTTCCTACAGCTCCTGGGCAACGTGCTGGTTATTGT | |
| | GCTGTCTCATCATTTTGGCAAAGAATTGCAAGTTTGTAC | |
| | AAAAAAGCAGGCTGCCACCATGGTGAGCAAGGGCGAG | |
| | GAGCTGTTCACCGGGGTGGTGCCCATCCTGGTCGAGC | |
| | TGGACGGCGACGTAAACGGCCACAAGTTCAGCGTGTC | |
| | CGGCGAGGGCGAGGGCGATGCCACCTACGGCAAGCT | |
| | GACCCTGAAGTTCATCTGCACCACCGGCAAGCTGCCCG | |
| | TGCCCTGGCCCACCCTCGTGACCACCCTGACCTACGG | |
| | CGTGCAGTGCTTCAGCCGCTACCCCGACCACATGAAGC | |
| | AGCACGACTTCTTCAAGTCCGCCATGCCCGAAGGCTAC | |
| | GTCCAGGAGCGCACCATCTTCTTCAAGGACGACGGCAA | |
| | CTACAAGACCCGCGCCGAGGTGAAGTTCGAGGGCGAC | |
| | ACCCTGGTGAACCGCATCGAGCTGAAGGGCATCGACTT | |
| | CAAGGAGGACGGCAACATCCTGGGGCACAAGCTGGAG | |
| | TACAACTACAACAGCCACAACGTCTATATCATGGCCGA | |
| | CAAGCAGAAGAACGGCATCAAGGTGAACTTCAAGATCC | |
| | GCCACAACATCGAGGACGGCAGCGTGCAGCTCGCCGA | |
| | CCACTACCAGCAGAACACCCCCATCGGCGACGGCCCC | |
| | GTGCTGCTGCCCGACAACCACTACCTGAGCACCCAGTC | |
| | CGCCCTGAGCAAAGACCCCAACGAGAAGCGCGATCAC | |
| | ATGGTCCTGCTGGAGTTCGTGACCGCCGCCGGGATCA | |
| | CTCTCGGCATGGACGAGCTGTACAAGTAAACCCAGCTT | |
| | TCTTGTACAAAGTGGTGATGGCCGGCCGCTTCGAGCAG | |
| | ACATGATAAGATACATTGATGAGTTTGGACAAACCACAA | |
| | CTAGAATGCAGTGAAAAAAATGCTTTATTTGTGAAATTT | |
| | GTGATGCTATTGCTTTATTTGTAACCATTATAAGCTGCA | |
| | ATAAACAAGTTAACAACAACAATTGCATTCATTTTATGTT | |
| | TCAGGTTCAGGGGGAGGTGTGGGAGGTTTTTTAAAGCA | |
| | AGTAAAACCTCTACAAATGTGGTAATCGATAGATCTAGG | |
| | AACCCCTAGTGATGGAGTTGGCCACTCCCTCTCTGCGC | |
| | GCTCGCTCGCTCACTGAGGCCGGGCGACCAAAGGTCG | |
| | CCCGACGCCCGGGCTTTGCCCGGGCGGCCTCAGTGAG | |
| | CGAGCGAGCGCGCAGCTGCCTGCAGGCAGCTTGGCAC | |
| | TGGCCGTCGTTTTACAACGTCGTGACTGGGAAAACCCT | |
| | GGCGTTACCCAACTTAATCGCCTTGCAGCACATCCCCC | |
| | TTTCGCCAGCTGGCGTAATAGCGAAGAGGCCCGCACC | |
| | GATCGCCCTTCCCAACAGTTGCGCAGCCTGAATGGCGA | |
| | ATGGCGCCTGATGCGGTATTTTCTCCTTACGCATCTGT | |
| | GCGGTATTTCACACCGCATACGTCAAAGCAACCATAGT | |
| | ACGCGCCCTGTAGCGGCGCATTAAGCGCGGCGGGTGT | |
| | GGTGGTTACGCGCAGCGTGACCGCTACACTTGCCAGC | |
| | GCCCTAGCGCCCGCTCCTTTCGCTTTCTTCCCTTCCTTT | |
| | CTCGCCACGTTCGCCGGCTTTCCCCGTCAAGCTCTAAA | |
| | TCGGGGGCTCCCTTTAGGGTTCCGATTTAGTGCTTTAC | |
| | GGCACCTCGACCCCAAAAAACTTGATTTGGGTGATGGT | |
| | TCACGTAGTGGGCCATCGCCCTGATAGACGGTTTTTCG | |
| | CCCTTTGACGTTGGAGTCCACGTTCTTTAATAGTGGACT | |
| | CTTGTTCCAAACTGGAACAACACTCAACCCTATCTCGG | |
| | GCTATTCTTTTGATTTATAAGGGATTTTGCCGATTTCGG | |
| | CCTATTGGTTAAAAAATGAGCTGATTTAACAAAAATTTAA | |
| | CGCGAATTTTAACAAAATATTAACGTTTACAATTTTATGG | |
| | TGCACTCTCAGTACAATCTGCTCTGATGCCGCATAGTTA | |
| | AGCCAGCCCCGACACCCGCCAACACCCGCTGACGCGC | |
| | CCTGACGGGCTTGTCTGCTCCCGGCATCCGCTTACAGA | |
| | CAAGCTGTGACCGTCTCCGGGAGCTGCATGTGTCAGA | |
| | GGTTTTCACCGTCATCACCGAAACGCGCGAGACGAAAG | |
| | GGCCTCGTGATACGCCTATTTTTATAGGTTAATGTCATG | |
| | ATAATAATGGTTTCTTAGACGTCAGGTGGCACTTTTCGG | |
| | GGAAATGTGCGCGGAACCCCTATTTGTTTATTTTTCTAA | |
| | ATACATTCAAATATGTATCCGCTCATGAGACAATAACCC | |
| | TGATAAATGCTTCAATAATATTGAAAAAGGAAGAGTATG | |
| | AGTATTCAACATTTCCGTGTCGCCCTTATTCCCTTTTTTG | |
| | CGGCATTTTGCCTTCCTGTTTTTGCTCACCCAGAAACGC | |
| | TGGTGAAAGTAAAAGATGCTGAAGATCAGTTGGGTGCA | |
| | CGAGTGGGTTACATCGAACTGGATCTCAACAGCGGTAA | |
| | GATCCTTGAGAGTTTTCGCCCCGAAGAACGTTTTCCAAT | |
| | GATGAGCACTTTTAAAGTTCTGCTATGTGGCGCGGTATT | |
| | ATCCCGTATTGACGCCGGGCAAGAGCAACTCGGTCGC | |
| | CGCATACACTATTCTCAGAATGACTTGGTTGAGTACTCA | |
| | CCAGTCACAGAAAAGCATCTTACGGATGGCATGACAGT | |
| | AAGAGAATTATGCAGTGCTGCCATAACCATGAGTGATAA | |
| | CACTGCGGCCAACTTACTTCTGACAACGATCGGAGGAC | |
| | CGAAGGAGCTAACCGCTTTTTTGCACAACATGGGGGAT | |
| | CATGTAACTCGCCTTGATCGTTGGGAACCGGAGCTGAA | |
| | TGAAGCCATACCAAACGACGAGCGTGACACCACGATGC | |
| | CTGTAGCAATGGCAACAACGTTGCGCAAACTATTAACT | |
| | GGCGAACTACTTACTCTAGCTTCCCGGCAACAATTAATA | |
| | GACTGGATGGAGGCGGATAAAGTTGCAGGACCACTTCT | |
| | GCGCTCGGCCCTTCCGGCTGGCTGGTTTATTGCTGATA | |
| | AATCTGGAGCCGGTGAGCGTGGGTCTCGCGGTATCATT | |
| | GCAGCACTGGGGCCAGATGGTAAGCCCTCCCGTATCG | |
| | TAGTTATCTACACGACGGGGAGTCAGGCAACTATGGAT | |
| | GAACGAAATAGACAGATCGCTGAGATAGGTGCCTCACT | |
| | GATTAAGCATTGGTAACTGTCAGACCAAGTTTACTCATA | |
| | TATACTTTAGATTGATTTAAAACTTCATTTTTAATTTAAAA | |
| | GGATCTAGGTGAAGATCCTTTTTGATAATCTCATGACCA | |
| | AAATCCCTTAACGTGAGTTTTCGTTCCACTGAGCGTCAG | |
| | ACCCCGTAGAAAAGATCAAAGGATCTTCTTGAGATCCTT | |
| | TTTTTCTGCGCGTAATCTGCTGCTTGCAAACAAAAAAAC | |
| | CACCGCTACCAGCGGTGGTTTGTTTGCCGGATCAAGAG | |
| | CTACCAACTCTTTTTCCGAAGGTAACTGGCTTCAGCAGA | |
| | GCGCAGATACCAAATACTGTTCTTCTAGTGTAGCCGTA | |
| | GTTAGGCCACCACTTCAAGAACTCTGTAGCACCGCCTA | |
| | CATACCTCGCTCTGCTAATCCTGTTACCAGTGGCTGCT | |
| | GCCAGTGGCGATAAGTCGTGTCTTACCGGGTTGGACTC | |
| | AAGACGATAGTTACCGGATAAGGCGCAGCGGTCGGGC | |
| | TGAACGGGGGGTTCGTGCACACAGCCCAGCTTGGAGC | |
| | GAACGACCTACACCGAACTGAGATACCTACAGCGTGAG | |
| | CTATGAGAAAGCGCCACGCTTCCCGAAGGGAGAAAGG | |
| | CGGACAGGTATCCGGTAAGCGGCAGGGTCGGAACAGG | |
| | AGAGCGCACGAGGGAGCTTCCAGGGGGAAACGCCTGG | |
| | TATCTTTATAGTCCTGTCGGGTTTCGCCACCTCTGACTT | |
| | GAGCGTCGATTTTTGTGATGCTCGTCAGGGGGGCGGA | |
| | GCCTATGGAAAAACGCCAGCAACGCGGCCTTTTTACGG | |
| | TTCCTGGCCTTTTGCTGGCCTTTTGCTCACATGTTCTTT | |
| | CCTGCGTTATCCCCTGATTCTGTGGATAACCGTATTACC | |
| | GCCTTTGAGTGAGCTGATACCGCTCGCCGCAGCCGAA | |
| | CGACCGAGCGCAGCGAGTCAGTGAGCGAGGAAGCGG | |
| | AAGAGCGCCCAATACGCAAACCGCCTCTCCCCGCGCG | |
| | TTGGCCGATTCATTAATGCAGCTGGCACGACAGGTTTC | |
| | CCGACTGGAAAGCGGGCAGTGAGCGCAACGCAATTAA | |
| | TGTGAGTTAGCTCACTCATTAGGCACCCCAGGCTTTAC | |
| | ACTTTATGCTTCCGGCTCGTATGTTGTGTGGAATTGTGA | |
| | GCGGATAACAATTTCACACAGGAAACAGCTATGACCAT | |
| | GATTACGAATTGCCTGCAGGCAG | |
| 31 | TTGCCCCAGGCCAGGACACAATGCGGGCGG | Murine GSDME |
| | | sgRNA 1 |
| 32 | CGTGAAAGAACGGAGCGTGCAGGACGGAGA | Murine GSDME |
| | | sgRNA 2 |
| 33 | GTGCAGGACGGAGAGGCGGCGCGCGGGTAA | Murine GSDME |
| | | sgRNA 3 |
| 34 | GAGAGGCGGCGCGCGGGTAAGTGGCGGCGG | Murine GSDME |
| | | sgRNA 4 |
| 35 | GGGCGGAGGGAAGAGGAGCCCAAGCGGAAC | Murine GSDME |
| | | sgRNA 5 |
| 36 | GGGGAGCGACGGGACCTGCCGTTCCGGCGG | Human GSDME |
| | | sgRNA 1 |
| 37 | GGCTCCTCCGACTCCGCCAGCGCAGGGGGC | Human GSDME |
| | | sgRNA 1 |
| 38 | AGGCTCCTCCGACTCCGCCAGCGCAGGGGG | Human GSDME |
| | | sgRNA 3 |
| 39 | CTCCTCCGACTCCGCCAGCGCAGGGGGCGC | Human GSDME |
| | | sgRNA 4 |
| 40 | CCGGGGACCCAGCAGCGGGCGCGCGGGTAA | Human GSDME |
| | | sgRNA 5 |

It should be emphasized that the above-described embodiments are merely examples of possible implementations. Many variations and modifications may be made to the above-described embodiments without departing from the principles of the present disclosure. All such modifications and variations are intended to be included herein within the scope of this disclosure and protected by the following claims.

At least the following is claimed:

1) A composition for knocking down or otherwise reducing Gasdermin E activity, comprising:
one or more nucleotides targeting Gasdermin E, one or more small molecules targeting or otherwise selective for Gasdermin E, or any combination of any thereof, wherein the one or more nucleotides targeting Gasdermin E are one or more anti-Gasdermin E shRNAs, siRNAs, or CRISPRi sgRNAs.

2) The composition of claim 1, wherein the one or more anti-Gasdermin E shRNAs comprise any one or more nucleotides with at least 85% sequence identity with any one or more of SEQ ID NOs: 1-19, individually or in any combination of any thereof.

3) The composition of claim 1, wherein the one or more anti-Gasdermin E siRNAs comprise any one or more nucleotides with at least 85% sequence identity with any one or more of SEQ ID NOs: 20-29, individually or in any combination of any thereof.

4) The composition of claim 1, wherein the one or more anti-Gasdermin E CRISPRi sgRNAs comprise any one or more nucleotides with at least 85% sequence identity with any one or more of SEQ ID NOs: 31-40, individually or in any combination of any thereof.

5) The composition of claim 1, wherein the one or more small molecules targeting or otherwise selective for Gasdermin E comprise any one or more of CuET (copper diethyldithiocarbamate), Disulfiram 20-hydroxyecdysone, Ac-DMPD-CMK, Ac-DMLD-CMK, Dimethyl Fumarate (DMF), or 2-bromopalmitate (2-BP), individually or in any combination of any thereof.

6) A pharmaceutical composition, comprising:
a composition of claim 1; and
a pharmaceutically-acceptable carrier.

7) A method of reducing Gasdermin E activity, comprising:
administering a composition of claim 1 to a subject in need thereof.

8) The method of claim 7, wherein the subject in need thereof is a subject having or suspected of having one or more of Traumatic Optic Neuropathy (TON), Glaucoma, juvenile glaucoma, Leber's Hereditary Optic Neuropathy, Optic Neuritis, Ischemic Optic Neuropathy, Diabetic Retinopathy, Retinal Occlusion, Anterior Ischemic Optic Neuropathy (AION), Infectious Neuropathies, Neuromyelitis optica (NMO), Behçet's disease, optic nerve hypoplasia, Central or branch retinal artery occlusion (CRAO, BRAO), Radiation Optic Neuropathy, Optic nerve glioma, or Age-related macular degeneration (AMD), individually or in any combination of any thereof.

9) A method of reducing neuronal cell death, comprising
administering a composition of claim 1 to a subject in need thereof.

10) The method of claim 9, wherein the subject in need thereof is a subject having or suspected of having one or more of Traumatic Optic Neuropathy (TON), Glaucoma, juvenile glaucoma, Leber's Hereditary Optic Neuropathy, Optic Neuritis, Ischemic Optic Neuropathy, Diabetic Retinopathy, Retinal Occlusion, Anterior Ischemic Optic Neuropathy (AION), Infectious Neuropathies, Neuromyelitis optica (NMO), Behçet's disease, optic nerve hypoplasia, Central or branch retinal artery occlusion (CRAO, BRAO), Radiation Optic Neuropathy, Optic nerve glioma, or Age-related macular degeneration (AMD), individually or in any combination of any thereof.

11) The method of claim 9, wherein the neuronal cell death comprises retinal ganglion cell death in the subject.

12) A method of reducing axonal degeneration, comprising:
administering a composition of claim 1 to a subject in need thereof.

13) The method of claim 12, wherein the subject in need thereof is a subject having or suspected of having one or more of Traumatic Optic Neuropathy (TON), Glaucoma, juvenile glaucoma, Leber's Hereditary Optic Neuropathy, Optic Neuritis, Ischemic Optic Neuropathy, Diabetic Retinopathy, Retinal Occlusion, Anterior Ischemic Optic Neuropathy (AION), Infectious Neuropathies, Neuromyelitis optica (NMO), Behçet's disease, optic nerve hypoplasia, Central or branch retinal artery occlusion (CRAO, BRAO), Radiation Optic Neuropathy, Optic nerve glioma, or Age-related macular degeneration (AMD), individually or in any combination of any thereof.

14) The method of claim 12, wherein the axonal degeneration comprises retinal ganglion cell axonal degeneration.

15) A method treating a disorder with retinal ganglion cell death and/or axonal degeneration, comprising:
administering a composition of claim 1 to a subject in need thereof.

16) The method of claim 15, wherein the subject in need thereof is a subject having or suspected of having one or more of Traumatic Optic Neuropathy (TON), Glaucoma, juvenile glaucoma, Leber's Hereditary Optic Neuropathy, Optic Neuritis, Ischemic Optic Neuropathy, Diabetic Retinopathy, Retinal Occlusion, Anterior Ischemic Optic Neuropathy (AION), Infectious Neuropathies, Neuromyelitis optica (NMO), Behçet's disease, optic nerve hypoplasia, Central or branch retinal artery occlusion (CRAO, BRAO), Radiation Optic Neuropathy, Optic nerve glioma, or Age-related macular degeneration (AMD), individually or in any combination of any thereof.

17) The method of claim 15, wherein the axonal degeneration comprises retinal ganglion cell axonal degeneration.

18) A kit, comprising:
a composition of claim 1; and
instructions for use.

19) The kit of claim 18, wherein the composition is provided in a dose unit form.


SEQUENCE LISTING (40 sequences)

SEQ ID NO: 1 - RNA, 21 nt, synthetic construct
  source 1..21: mol_type = other RNA
gatgatggagtatctgatctt

SEQ ID NO: 2 - RNA, 21 nt, synthetic construct
  source 1..21: mol_type = other RNA
gggatccagaccaagactata

SEQ ID NO: 3 - RNA, 21 nt, synthetic construct
  source 1..21: mol_type = other RNA
gtcaccacggacaccaatgta

SEQ ID NO: 4 - RNA, 21 nt, synthetic construct
  source 1..21: mol_type = other RNA
ggtcagcgcactagcagaaat

SEQ ID NO: 5 - RNA, 21 nt, synthetic construct
  source 1..21: mol_type = other RNA
gaatgactctgataagttaca

SEQ ID NO: 6 - DNA, 21 bp, synthetic construct
  source 1..21: mol_type = other DNA
gtgaaatacgagggcaagttt

SEQ ID NO: 7 - RNA, 21 nt, synthetic construct
  source 1..21: mol_type = other RNA
gacccagcatctgagttaggt

SEQ ID NO: 8 - RNA, 21 nt, synthetic construct
  source 1..21: mol_type = other RNA
gatgatggagtatctgatctt

SEQ ID NO: 9 - RNA, 21 nt, synthetic construct
  source 1..21: mol_type = other RNA
gcggtcctatttgatgatgaa

SEQ ID NO: 10 - RNA, 21 nt, synthetic construct
  source 1..21: mol_type = other RNA
gcatgatgaatgacctgactt

SEQ ID NO: 11 - RNA, 21 nt, synthetic construct
  source 1..21: mol_type = other RNA
gcttctaagtctggtgacaaa

SEQ ID NO: 12 - RNA, 21 nt, synthetic construct
  source 1..21: mol_type = other RNA
gcattcatagacatgccagat

SEQ ID NO: 13 - RNA, 21 nt, synthetic construct
  source 1..21: mol_type = other RNA
ctctgccgagagaacaataaa

SEQ ID NO: 14 - RNA, 21 nt, synthetic construct
  source 1..21: mol_type = other RNA
tctgccgagagaacaataaat

SEQ ID NO: 15 - RNA, 21 nt, synthetic construct
  source 1..21: mol_type = other RNA
agccaccaggtactctttaaa

SEQ ID NO: 16 - RNA, 21 nt, synthetic construct
  source 1..21: mol_type = other RNA
gaaagagtctgaccctttaat

SEQ ID NO: 17 - RNA, 21 nt, synthetic construct
  source 1..21: mol_type = other RNA
tggaagtgatgtatgttattt

SEQ ID NO: 18 - RNA, 21 nt, synthetic construct
  source 1..21: mol_type = other RNA
taaatgctcctagagtatatt

SEQ ID NO: 19 - RNA, 21 nt, synthetic construct
  source 1..21: mol_type = other RNA
gtattctgggtattctattaa

SEQ ID NO: 20 - RNA, 19 nt, synthetic construct
  source 1..19: mol_type = other RNA
tctgtctgatgatggagta

SEQ ID NO: 21 - RNA, 19 nt, synthetic construct
  source 1..19: mol_type = other RNA
tgacattagtctggagaga

SEQ ID NO: 22 - RNA, 19 nt, synthetic construct
  source 1..19: mol_type = other RNA
gaatgactctgataagtta

SEQ ID NO: 23 - RNA, 19 nt, synthetic construct
  source 1..19: mol_type = other RNA
cctacggtgtcattgagtt

SEQ ID NO: 24 - RNA, 19 nt, synthetic construct
  source 1..19: mol_type = other RNA
gcttctaagtctggtgaca

SEQ ID NO: 25 - RNA, 19 nt, synthetic construct
  source 1..19: mol_type = other RNA
ccttggcgatgtactcata

SEQ ID NO: 26 - RNA, 19 nt, synthetic construct
  source 1..19: mol_type = other RNA
gcagaagtgtgtgatctct

SEQ ID NO: 27 - RNA, 19 nt, synthetic construct
  source 1..19: mol_type = other RNA
tgtgtgatctctgagcaca

SEQ ID NO: 28 - RNA, 19 nt, synthetic construct
  source 1..19: mol_type = other RNA
gcttcgtgctctgtctgat

SEQ ID NO: 29 - RNA, 19 nt, synthetic construct
  source 1..19: mol_type = other RNA
ttagagaagttgatgctga

SEQ ID NO: 30 - DNA, 6497 bp, synthetic construct
  source 1..6497: mol_type = other DNA
ctgcgcgctcgctcgctcactgaggccgcccgggcaaagcccgggcgtcgggcgacctttggtcgcccggcctcagtgagcgagcgagcgcgcagagagggagtggccaactccatcactaggggttcctatcgatgagggcctatttcccatgattccttcatatttgcatatacgatacaaggctgttagagagataattggaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacaccggccgtcaagagaacagttaatactcgagtattaactgttctcttgacggtttttgaattccaactttgtatagaaaagttgctcgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtcgaggtgagccccacgttctgcttcactctccccatctcccccccctccccacccccaattttgtatttatttattttttaattattttgtgcagcgatgggggcggggggggggggggggcgcgcgccaggcggggcggggcggggcgaggggcggggcggggcgaggcggagaggtgcggcggcagccaatcagagcggcgcgctccgaaagtttccttttatggcgaggcggcggcggcggcggccctataaaaagcgaagcgcgcggcgggcgggagtcgctgcgcgctgccttcgccccgtgccccgctccgccgccgcctcgcgccgcccgccccggctctgactgaccgcgttactcccacaggtgagcgggcgggacggcccttctcctccgggctgtaattagcgcttggtttaatgacggcttgtttcttttctgtggctgcgtgaaagccttgaggggctccgggagggccctttgtgcggggggagcggctcggggggtgcgtgcgtgtgtgtgtgcgtggggagcgccgcgtgcggctccgcgctgcccggcggctgtgagcgctgcgggcgcggcgcggggctttgtgcgctccgcagtgtgcgcgaggggagcgcggccgggggcggtgccccgcggtgcggggggggctgcgaggggaacaaaggctgcgtgcggggtgtgtgcgtgggggggtgagcagggggtgtgggcgcgtcggtcgggctgcaaccccccctgcacccccctccccgagttgctgagcacggcccggcttcgggtgcggggctccgtacggggcgtggcgcggggctcgccgtgccgggcggggggtggcggcaggtgggggtgccgggcggggcggggccgcctcgggccggggagggctcgggggaggggcgcggcggcccccggagcgccggcggctgtcgaggcgcggcgagccgcagccattgccttttatggtaatcgtgcgagagggcgcagggacttcctttgtcccaaatctgtgcggagccgaaatctgggaggcgccgccgcaccccctctagcgggcgcggggcgaagcggtgcggcgccggcaggaaggaaatgggcggggagggccttcgtgcgtcgccgcgccgccgtccccttctccctctccagcctcggggctgtccgcggggggacggctgccttcgggggggacggggcagggcggggttcggcttctggcgtgtgaccggcggctctagagcctctgctaaccatgttcatgccttcttctttttcctacagctcctgggcaacgtgctggttattgtgctgtctcatcattttggcaaagaattgcaagtttgtacaaaaaagcaggctgccaccatggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaaacccagctttcttgtacaaagtggtgatggccggccgcttcgagcagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtaatcgatagatctaggaacccctagtgatggagttggccactccctctctgcgcgctcgctcgctcactgaggccgggcgaccaaaggtcgcccgacgcccgggctttgcccgggcggcctcagtgagcgagcgagcgcgcagctgcctgcaggcagcttggcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggcgcctgatgcggtattttctccttacgcatctgtgcggtatttcacaccgcatacgtcaaagcaaccatagtacgcgccctgtagcggcgcattaagcgcggcgggtgtggtggttacgcgcagcgtgaccgctacacttgccagcgccctagcgcccgctcctttcgctttcttcccttcctttctcgccacgttcgccggctttccccgtcaagctctaaatcgggggctccctttagggttccgatttagtgctttacggcacctcgaccccaaaaaacttgatttgggtgatggttcacgtagtgggccatcgccctgatagacggtttttcgccctttgacgttggagtccacgttctttaatagtggactcttgttccaaactggaacaacactcaaccctatctcgggctattcttttgatttataagggattttgccgatttcggcctattggttaaaaaatgagctgatttaacaaaaatttaacgcgaattttaacaaaatattaacgtttacaattttatggtgcactctcagtacaatctgctctgatgccgcatagttaagccagccccgacacccgccaacacccgctgacgcgccctgacgggcttgtctgctcccggcatccgcttacagacaagctgtgaccgtctccgggagctgcatgtgtcagaggttttcaccgtcatcaccgaaacgcgcgagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgcctacatacctcgctctgctaatcctgttaccagtggctgctgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgaattgcctgcaggcag

SEQ ID NO: 31 - RNA, 30 nt, synthetic construct
  source 1..30: mol_type = other RNA
ttgccccaggccaggacacaatgcgggcgg

SEQ ID NO: 32 - RNA, 30 nt, synthetic construct
  source 1..30: mol_type = other RNA
cgtgaaagaacggagcgtgcaggacggaga

SEQ ID NO: 33 - RNA, 30 nt, synthetic construct
  source 1..30: mol_type = other RNA
gtgcaggacggagaggcggcgcgcgggtaa

SEQ ID NO: 34 - RNA, 30 nt, synthetic construct
  source 1..30: mol_type = other RNA
gagaggcggcgcgcgggtaagtggcggcgg

SEQ ID NO: 35 - RNA, 30 nt, synthetic construct
  source 1..30: mol_type = other RNA
gggcggagggaagaggagcccaagcggaac

SEQ ID NO: 36 - RNA, 30 nt, synthetic construct
  source 1..30: mol_type = other RNA
ggggagcgacgggacctgccgttccggcgg

SEQ ID NO: 37 - RNA, 30 nt, synthetic construct
  source 1..30: mol_type = other RNA
ggctcctccgactccgccagcgcagggggc

SEQ ID NO: 38 - RNA, 30 nt, synthetic construct
  source 1..30: mol_type = other RNA
aggctcctccgactccgccagcgcaggggg

SEQ ID NO: 39 - RNA, 30 nt, synthetic construct
  source 1..30: mol_type = other RNA
ctcctccgactccgccagcgcagggggcgc

SEQ ID NO: 40 - RNA, 30 nt, synthetic construct
  source 1..30: mol_type = other RNA
ccggggacccagcagcgggcgcgcgggtaa